(19) United States
(12) Patent Application Publication
(10) Pub. No.: US 2026/0317835 A1
(43) Pub. Date: Oct. 15, 2026

(54) DETECTION AND CLASSIFICATION OF B-CELL MALIGNANCIES

(71) Applicants: DANA-FARBER CANCER INSTITUTE, INC., Boston, MA (US); THE GENERAL HOSPITAL CORPORATION, Boston, MA (US)

(72) Inventors: Irene M. Ghobrial, Boston, MA (US); Romanos Sklavenitis-Pistofidis, Jamaica Plain, MA (US); Gad Getz, Belmont, MA (US)

(21) Appl. No.: 19/683,065

(22) Filed: May 20, 2026

Related U.S. Application Data
(63) Continuation of application No. PCT/US2024/058982, filed on Dec. 6, 2024.
(60) Provisional application No. 63/607,961, filed on Dec. 8, 2023.

(51) Int. Cl. C12Q 1/6886; G16B 40/00

(52) U.S. Cl. CPC C12Q 1/6886; G16B 40/00; C12Q 2600/156; C12Q 2600/158

(57) ABSTRACT

There are provided methods for detecting and classifying a B-cell malignancy selected from the group consisting of a B-cell lymphoma, a B-cell leukemia, and a plasma cell dyscrasia in a subject. The methods employ a tumor cell profiling algorithm to analyze single-cell RNA sequencing data obtained from a sample of, e.g., the subject's blood or bone marrow, enriched for a cell type comprising tumoral cells using a cell surface marker. This algorithm identifies one or more one or more expanded clonal cell populations and categorizes each of the one or more expanded clonal cell populations as malignant or pre-malignant tumor cell population if expression of one or more malignancy markers is detected. The one or more malignant or pre-malignant tumor cell population(s) can be analyzed using a statistical model or machine learning algorithm to classify the B-cell malignancy.

### FIELD

There are provided methods for detecting and classifying lymphoproliferative disorders, in particular B-cell malignancies selected from the group consisting of B-cell lymphomas, B-cell leukemias, and plasma cell dyscrasias. The methods use single-cell RNA sequencing data (including single-cell B cell receptor (BCR) V(D)J sequencing data) obtained, e.g., from circulating tumor cells enriched from blood samples.

### BACKGROUND

A number of diseases are characterized by the abnormal proliferation of lymphocytes, often in conjunction with elevated levels of immunoglobulin protein in the blood. These lymphoproliferative disorders, like other hematological neoplasms, are often further characterized by the presence of cytogenetic abnormalities, such as translocations and chromosome loss or gain. The detection of these changes is essential for diagnosis and prognosis as well as for monitoring disease progression. One example is the plasma cell malignancy Multiple Myeloma (MM) and its precursor conditions.

For example, patients with B-cell malignancies such as Multiple Myeloma (MM) and its precursor conditions, Monoclonal Gammopathy of Undetermined Significance (MGUS) and Smoldering Multiple Myeloma (SMM), have variable outcomes depending on the tumor's cytogenetic profile. In particular, patients with MM and translocation t(4;14), t(14;16), or t(14;20), or gain or amplification of chr1q (Amp1q) and deletion of chr17p (Del17p) have worse prognosis (i.e., so-called high-risk MM) compared to patients with translocation t(11;14) or hyperdiploidy (HRD). Similarly, patients with SMM and t(4;14), Amp1q and deletion of chr13q (Del13q) may be more likely to progress to full-blown MM.

Furthermore, a lymphoproliferative tumor's cytogenetic profile may alter patient management. For example, MM patients with high-risk cytogenetic abnormalities may receive a quadruplet combination therapy (daratumumab, lenalidomide, bortezomib, and dexamethasone) instead of a triplet combination therapy (lenalidomide, bortezomib, and dexamethasone), followed by autologous stem cell transplantation (ASCT) and maintenance with both bortezomib and lenalidomide. MM patients with standard-risk cytogenetic abnormalities, e.g., chromosomal translocation t(11;14) may avoid ASCT and receive maintenance therapy with lenalidomide alone. Moreover, MM patients with chromosomal translocation t(11;14) show superior response to the BCL-2 inhibitor Venetoclax.

Clinically, a lymphoproliferative tumor's cytogenetic profile can be determined by Fluorescence In Situ Hybridization (FISH) and karyotyping. In patients suffering from MM, karyotyping fails completely in most cases, as malignant plasma cells (i.e., myeloma cells) do not proliferate ex vivo, and it is also suboptimal for the detection of myeloma translocations which are typically cryptic in metaphase karyotypes. While FISH does not require cells to proliferate ex vivo, it still is limited by the number of tumor cells that can be detected. In MM diagnosis, FISH is typically paired with cytoplasmic staining for immunoglobulin, which enables the clinical laboratory to focus its analysis on plasma cells that share the light chain of the tumor (for example, lambda-restricted plasma cells in a patient with IgG Lambda MM). Despite that, FISH returns inconclusive results in a large fraction of patients with MM. FISH is even less sensitive in patients with precursor disease, such as MGUS and SMM, which commonly show reduced numbers of tumor cells in the bone marrow (BM).

FISH and karyotyping are performed on cells acquired from a bone marrow biopsy, a sampling method that may be avoided by patients and physicians alike as it is invasive and can be painful. Additionally, bone marrow biopsies are also susceptible to dilution by blood when obtained by aspiration, reducing the representation of tumor cell populations within the in situ tissue. Moreover, MM in particular is a multifocal, spatially heterogeneous disease, where a single-site BM biopsy alone may not provide comprehensive information on tumor burden as well as the underlying disease biology occurring throughout the body.

As a result, many patients may be cytogenetically unclassified and repeated testing for updated profiling may not be possible. However, updating the patient's cytogenetic profile over time can be important, as many high-risk genomic abnormalities are secondary, i.e., can be acquired or evolve over time.

The use of blood biopsies could potentially avoid the need for painful and risky bone marrow biopsies in patients suffering from lymphoproliferative disorders. They are minimally invasive, which also decreases the financial costs and potential complications associated with tissue biopsies. Moreover, they are easy to iterate during patients' follow-up. Both features are crucial, considering how cancer cells adapt to pharmacological pressures, and acquire new molecular alterations not detected at baseline.

Isolated circulating tumor cells (CTCs) can be used to conduct a higher resolution molecular characterization of various cancers. For example, tumor-associated genetic abnormalities can be identified using whole genome sequencing (WGS) in CTCs. However, WGS commonly requires thousands of cells, and even with newly developed low-input library preparation technology, requires at least 50 CTCs. CTCs are rare in peripheral blood (estimated at 1-10 CTCs out of 7,000,000 nucleated cells. Extensive enrichment for CTCs is thus commonly required before WGS can be performed. As a direct correlation is known to exist between higher CTC number and poor clinical outcomes, much effort has been applied to the optimization of methods for enrichment and isolation of CTCs from peripheral blood.

More recently, the use of single-cell RNA sequencing has been explored as a diagnostic modality for cancer. As with WGS, however, single-cell RNA sequencing requires large sample sizes. Moreover, background noise associated with single-cell RNA sequencing data is significant and represents an additional hurdle. Genes that are not very highly expressed, for example transcription factors which are frequently involved in translocations in lymphoproliferative disorders, are not well captured by single-cell RNA sequencing, and so tumor cells that do express them may yet falsely appear to show zero expression. Furthermore, single-cell RNA sequencing cannot directly detect the presence of immunoglobulin heavy chain (IgH) translocations which do not result in fusion transcripts and which are common in lymphoproliferative disorders. For these reasons, single-cell RNA sequencing has not been adopted for cancer diagnostics in lymphoproliferative disorders or otherwise.

Therefore, a need remains for new diagnostic methods for patients with known or suspected MM as well as other lymphoproliferative disorders such as other plasma cell dyscrasias, B-cell lymphomas, and B-cell leukemias, and for risk assessment, diagnosis, and monitoring disease progression or assessing the effectiveness of therapeutic interventions.

### SUMMARY

There are provided highly sensitive and accurate methods for detecting and characterizing lymphoproliferative disorders, in particular B-cell malignancies such as B-cell lymphomas, B-cell leukemias, and plasma cell dyscrasias. The methods address challenges associated with the small number of tumor cells that may be isolated from bone marrow or blood biopsies. In particular, the methods provided herein combine enrichment of tumor cells such as circulating tumor cells (CTCs) with computational methods which enable the identification and cytogenic profiling of such disorders with high accuracy.

Specifically, single-cell RNA sequencing is used to overcome the long-standing challenges of high-throughput detection and recovery of rare CTCs, even in subjects with few CTCs, without necessitating sophisticated enrichment steps upstream. This is especially beneficial during the early stages of many B-cell malignancies when few CTCs are found in the blood circulation. Moreover, the described methods provide a level of tumor characterization that normally requires several separate assays (e.g., FISH for cytogenetics, and microarrays or bulk RNA sequencing for gene expression profiling), which typically necessitate an invasive bone marrow (BM) biopsy as input.

In particular, in one aspect, a method for detecting a B-cell malignancy selected from the group consisting of a B-cell lymphoma, a B-cell leukemia, and a plasma cell dyscrasia in a subject is provided that comprises:
I. enriching a sample obtained from the subject by selecting for a cell type comprising tumoral cells using a cell surface marker; and
II. performing the following analysis steps:
   a) obtaining single-cell RNA sequencing data including single-cell B cell receptor (BCR) V(D)J sequencing data from the cells of the enriched sample;
   b) applying a tumor cell profiling algorithm to the single-cell RNA sequencing data, wherein the algorithm:
      i. identifies cells expressing one or more lineage markers of the cell type comprising the tumoral cells;
      ii. identifies among the cells expressing the one or more lineage markers one or more one or more expanded clonal cell populations based on the BCR V(D)J sequencing data; and
      iii. categorizes each of the one or more expanded clonal cell populations as malignant or pre-malignant tumor cell population if expression of a pre-determined set of oncogenes and tumor surface markers specific to the B-cell malignancy is detected.

In another aspect, a computer-implemented method for classifying a B-cell malignancy selected from the group consisting of a B-cell lymphoma, a B-cell leukemia, and a plasma cell dyscrasia in a subject is provided that comprises:
a) obtaining single-cell RNA sequencing data including single-cell B cell receptor (BCR) V(D)J sequencing data for a population of cells, wherein the population of cells was obtained from a sample obtained from the subject by enriching the sample for a cell type comprising tumoral cells using a cell surface marker;
b) applying a tumor cell profiling algorithm to the single-cell RNA sequencing data; wherein the algorithm:
   (i) identifies cells expressing one or more lineage markers of the cell type comprising the tumoral cells;
   (ii) identifies among the cells expressing the one or more lineage markers one or more expanded clonal cell populations based on the BCR V(D)J sequencing data;
   (iii) categorizes each of the one or more expanded clonal cell populations as malignant or pre-malignant if expression of a pre-determined set of oncogenes and tumor surface markers specific to the B-cell malignancy is detected; and
   (iv) determines the expression of translocation partner genes and/or infers one or more copy number variants (CNVs) for the one or more malignant or pre-malignant clonal cell population(s) to classify the B-cell malignancy.

For the purposes of cytogenetic profiling, the algorithm may additionally determine the expression of translocation partner genes for the one or more malignant or pre-malignant tumor cell population(s). Moreover, the algorithm may further infer one or more copy number variants (CNVs) for the one or more malignant or pre-malignant clonal cell population(s).

In a further aspect, a method of treating a patient with a B-cell malignancy selected from the group consisting of a B-cell lymphoma, a B-cell leukemia, and a plasma cell dyscrasia is provided that comprises:
a) classifying or having classified the B-cell malignancy by the method of any of the preceding claims; and
b) selecting and administering a treatment based on the classification.

In yet a further aspect, a method of training a statistical model or machine learning algorithm to classify a B-cell malignancy selected from the group consisting of a B-cell lymphoma, a B-cell leukemia, and a plasma cell dyscrasia is provided that comprises:
a) cytogenetically classifying tumor tissue and/or circulating tumor cell (CTC) samples taken from subjects with the B-cell malignancy and optionally related B-cell malignancies, to thereby generate ground truth label data that identifies cytogenetic classifications of the tumor tissue and/or CTC samples;
b) carrying out single-cell RNA sequencing on cells from the tumor tissue and/or CTC samples, to thereby generate training input data; and
c) training the model or machine learning algorithm, using the training input data and the ground truth label data, to classify the B-cell malignancy.

The cytogenetic profile may comprise one or more translocations and/or one or more copy number abnormalities.

### BRIEF DESCRIPTION OF THE DRAWINGS

Further description, by way of example, is provided with reference to the following drawings.

FIGS. 1A and 1B are flowcharts illustrating an example of a method performed by a tumor cell profiling algorithm.

FIG. 2 is a flowchart illustrating an example of a method of training a statistical model.

FIG. 3 schematically illustrates a computer system upon which a computer program according to an embodiment, or any part thereof, may run.

FIG. 4 shows boxplots with percentage of malignant plasma cells relative to total malignant and non-malignant plasma cells (y-axis) in the peripheral blood of MM patients grouped by both disease stage and SMM risk status (x-axis), with subclassifications of newly diagnosed (NDMM), low-risk (LRSMM), intermediate-risk (IRSMM, or high-risk (HRSMM). P-values were adjusted using the Benjamini-Hochberg approach.

FIG. 5 shows a scatterplot demonstrating the correlation between plasma cells present within bone marrow aspirates compared to circulating tumor cells in the peripheral blood.

FIG. 6 shows the percentage of patients with MM or precursor conditions which were not cytogenetically classified by FISH.

FIG. 7 shows a split violin plot with expression levels of common IgH translocation partner genes present in bone marrow tumor cells and circulating tumor cells. Each split violin represents an individual patient.

FIG. 8 shows a confusion matrix visualizing the performance of 51 leave-one-out multiple myeloma cytogenetic classification models for each of the 51 hold-out cases. Each tile shows the absolute number of cases.

FIG. 9 shows a confusion matrix visualizing the performance of an aggregated bone marrow-trained multiple myeloma cytogenetic classification model on circulating tumor cell samples. Each tile shows the absolute number of cases.

FIG. 10 shows multiple myeloma patients identified as having the chromosomal events of Amp1q, Del13q and Del17p, as detected by FISH and/or by single-cell RNA sequencing methods as described herein. For each event, the bars show detection of the events using, from left to right, FISH and single-cell RNA sequencing; FISH only; and single-cell RNA sequencing only.

FIG. 11 shows a scatterplot of subclone frequency in 20 cases with both single-cell RNA seq (x-axis) and whole genome sequencing (y-axis). Cases in which the subclone was considered to be clonal (i.e., with no subclone detected) by whole genome sequencing are circled at the upper right corner.

FIG. 12 shows a heatmap of correct classifications of downsampled circulating tumor cell populations applied to the multiple myeloma cytogenetic classification model described in Example 6. Percentage correct classification is shown out of 10 iterations at various sample sizes.

FIG. 13 shows a scatterplot of circulating tumor cell samples correctly classified by the multiple myeloma cytogenetic classification model described in Example 6 compared to FISH classification across CTC sample sizes. Error bars correspond to 95% confidence intervals.

FIG. 14 shows a barplot of the number of circulating tumor cells detected in patient blood samples. Patients are colored by disease stage and patients with SMM have been subclassified into low-risk (LRSMM), intermediate-risk (IRSMM, or high-risk (HRSMM), using the International Myeloma Working Group criteria.

FIG. 15 shows a confusion matrix visualizing the performance of 134 leave-one-out multiple myeloma cytogenetic classification models for each of the 134 hold-out cases. Each tile shows the absolute number of cases.

FIG. 16 shows a confusion matrix visualizing the performance of an aggregated bone marrow-trained multiple myeloma cytogenetic classification model on circulating tumor cell samples. Each tile shows the absolute number of cases.

FIG. 17 shows a confusion matrix visualizing the performance of an aggregated bone marrow-trained multiple myeloma cytogenetic classification model on circulating tumor cell samples compared to research-level whole genome sequencing (WGS; n=23) and in cases unclassified by either FISH or WGS (n=37). Each tile shows the absolute number of cases.

### DETAILED DESCRIPTION

Unless otherwise defined herein, technical and scientific terms have the same meaning as commonly understood by one of ordinary skill in the art to which this application belongs and as commonly used in the art to which this application belongs. Exemplary methods and materials are described below, although methods and materials similar or equivalent to those described herein can also be used in the practice or testing of the present disclosure. In case of conflict, the present specification, including definitions, will control. To facilitate ready understanding, certain terms used herein are first defined below.

Unless otherwise required by context, singular terms shall include pluralities and plural terms shall include the singular. Thus, the singular forms “a”, “an”, and “the” include plural referents unless the context clearly dictates otherwise. For example, “a plasma cell” is understood to represent one or more plasma cell(s) or a population of plasma cells. As such, the terms “a” (or “an”), “one or more”, and “at least one” can be used interchangeably herein.

“And/or” is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is used interchangeably with “A and/or B and/or C” and is intended to encompass each of the following aspects: A, B, and C; A, B, or C; A or C; A or B; B or C; A and C; A and B; B and C; A (alone); B (alone); and C (alone).

The words “have” and “comprise”, or variations such as “has”, “having”, “comprises”, or “comprising” will be understood to imply the inclusion of a stated integer or group of integers but not the exclusion of any other integer or group of integers. It is further understood that wherever aspects are described herein with the language “comprising” or “having” or grammatical equivalents thereof, otherwise analogous aspects described in terms of “consisting of” and/or “consisting essentially of” are also provided. In other words, if a composition comprising A, B and C is recited, a composition consisting essentially of A, B and C is also contemplated as is a composition consisting of A, B and C.

The term “about” refers to an interval of accuracy that a person skilled in the art will understand to still ensure the technical effect of the feature in question. The term indicates a deviation from the indicated numerical value of =10, +5%, or +1%.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The terms “reverse transcription”, “reverse transcribed”, “RT-PCR” and similar terms refer to reverse transcription polymerase chain reaction, a process for amplifying RNA. RNA molecules are reverse transcribed to complementary DNA (cDNA) using reverse transcriptase and then using PCR to amplify the resulting cDNA.

The term “lymphoproliferative disorder” refers to a condition in which lymphocytes are produced in excessive quantities. Non-limiting examples of diseases include any B cell, T cell or plasma cell neoplasm.

The term “B-cell malignancy” refers to a condition in which antibody-producing cells such as B cells or plasma cells are produced in excessive quantities. Chromosomal translocations involving the immunoglobulin (Ig) loci are a hallmark of most such B cell malignancies. For example, the term includes B-cell lymphomas, B-cell leukemias, and plasma cell dyscrasias, as well as their precursor conditions.

The term “aneuploidy” refers to the context of a cell having an abnormal number of chromosomes relative to a cell of normal ploidy.

The term “disease” or “disorder” refers to any condition or disorder that damages or interferes with the normal function of a cell, tissue, or organ. The disease is any disease that may be characterized by enriching for or isolating circulating tumor cells (CTCs). In some instances, the disease is a hematological pre-malignancy or malignancy (e.g., a blood cancer), or other lymphoproliferative disease, e.g. a B-cell malignancy such as a B-cell leukemia, or a plasma cell dyscrasia (or a precursor condition thereof).

In some instances, steps that are performed by an algorithm refer to “a cell”, “cells”, or “cell populations”, and the like. It is understood that this is a reference to the underlying single-cell RNA sequencing data representing the transcriptome of the cell, cells, or cell population. Thus, if the algorithm identifies one or more expanded clonal cell populations, it identifies the one or more data sets within the single-cell RNA sequencing data that correspond to, or are representative of, the one or more expanded clonal cell populations. Similarly, when one or more expanded clonal cell populations are categorized as malignant or pre-malignant tumor cell population, the algorithm identifies data sets that correspond to, or are representative of, the one or more expanded clonal cell populations and identifies data sub-sets associated with expression values of one or more malignancy markers above a pre-determined threshold. Likewise, when one or more malignant or pre-malignant tumor cell population(s) are analyzed using a statistical model or machine learning algorithm, the analysis is performed on the data subsets corresponding to, or representative of, the one or more malignant or pre-malignant tumor cell population(s).

Where cells deriving from a lymphoproliferative disorder are discussed herein, such as circulating tumor cells, these cells may be described herein as ‘tumoral’ or ‘malignant’, even if the underlying condition may not be clinically described as a tumor or malignancy.

Generally, techniques of cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, nomenclature used in connection with these technology areas herein is as commonly used in the art as can be reference seen by to, e.g., http://www.informatics.jax.org/mgihome/nomen/gene.shtml and https://www.genenames.org/.

### Lymphoproliferative Disorders

There are provided methods for detecting a lymphoproliferative disorder such as a B-cell malignancy in a subject, in particular computer-implemented methods that additionally classify the lymphoproliferative disorder, e.g., by cytogenetically profiling tumor cells. The classification may include information about the disease stage. The classification can be used for disease prognosis or selection of a suitable therapy or treatment regimen.

Accordingly, a method of treating a patient with a lymphoproliferative disorder such as a B-cell malignancy is provided, wherein the method comprises (a) classifying the lymphoproliferative disorder by a method described herein, and (b) selecting and administering a treatment based on the classification.

The methods described herein can be used for diagnosing a lymphoproliferative disorder such as a B-cell malignancy (e.g., a B-cell lymphoma, a B-cell leukemia, or a plasma cell dyscrasia) in a subject. The methods can also be used to monitor a treatment response in a subject. The methods can further be used to monitor or screen a subject susceptible to developing a lymphoproliferative disorder such as a B-cell malignancy. The subject may have previously been diagnosed with a pre-malignant stage of the lymphoproliferative disorder.

### Types of Lymphoproliferative Disorders

Lymphoproliferative disorders include disorders of B-, T- and NK-cell lineages. Each lineage encompasses highly heterogenous groups of lymphoproliferative disorders and subtypes of such disorders. For example, different subtypes of B-cell non-Hodgkin lymphoma (B-NHL) can display different morphologies, phenotypes, and cytogenetic profiles, which require distinct clinical management. Accordingly, the accurate classification of a given lymphoproliferative disorder is necessary so that appropriate treatment can be provided.

Exemplary lymphoproliferative disorders include, but are not restricted to, lymphomas including B-cell and T-cell lymphomas, leukemias and plasma cell dyscrasias. Lymphomas may include mantle cell lymphoma, Burkitt lymphoma, diffuse large B cell lymphoma (DLBCL), small lymphocytic lymphoma, lymphoplasmacytic lymphoma, and follicular lymphoma. Leukemias may include chronic lymphocytic leukemia, acute lymphoblastic leukemia, hairy cell leukemia, and plasma cell leukemia.

Other lymphoproliferative disorder include Waldenstrom macroglobulinemia (WM); light-chain amyloidosis (AL); plasmacytoma (e.g., solitary plasmacytoma of bone, extramedullary plasmacytoma); light chain deposition disease; heavy-chain disease; Polyneuropathy, Organomegaly, Endocrinopathy, Monoclonal plasma cell disorder, Skin changes (POEMS) syndrome; hemophagocytic lymphohistiocytosis (HLH); Langerhans cell histiocytosis (LCH); autoimmune lymphoproliferative syndrome (ALPS); virus-associated conditions such as Epstein-Barr virus-associated lymphoproliferative diseases; hereditary conditions such as Wiskott-Aldrich syndrome and X-linked lymphoproliferative disease; and post-transplant lymphoproliferative disorder.

In particular, the provided methods are applicable to lymphoproliferative disorders such as B-cell leukemias, B-cell lymphomas, plasma cell dyscrasias, and myelomas, including any premalignant versions thereof. Plasma cell dyscrasias (e.g., monoclonal gammopathies) may include monoclonal gammopathy of undermined significance (MGUS), smoldering multiple myeloma (SMM), symptomatic multiple myeloma (MM), IgM MGUS, smoldering Waldenstrom's Macroglobulinemia (SWM), symptomatic Waldenstrom's Macroglobulinemia (WM), and plasma cell leukemia.

### B-Cell Malignancies

Many B-cell malignancies-such as B-cell lymphomas, B-cell leukemias, and plasma cell dyscrasias—are characterized by the abnormal proliferation of B-cells or plasma cell, often in conjunction with elevated levels of immunoglobulin protein in the blood. Such B-cell malignancies are often further characterized by the presence of cytogenetic abnormalities, such as chromosomal translocations and chromosome loss or gain.

Indeed, various B-cell malignancies are distinguishable based on the presence of specific translocations. For example, patients with follicular lymphoma typically have chromosomal translocation t(14;18)(q32;q21). Similarly, chromosomal translocation t(11;14)(q13;q32) is highly characteristic of mantel cell lymphoma, while patients with Burkitt lymphoma typically have chromosomal translocation t(8;14)(q24;q32)(Grau et al. Best practice & research Clinical haematology, (2023)36(4): 101513). Multiple myeloma (MM) can be characterized by a number of different chromosomal translocations, including t(11;14), t(4;14), t(14;16), t(14;20), t(8;14), and t(6;14).

Accordingly, the detection of cytogenetic changes such as chromosomal translocations is especially useful in the detection and classification of some lymphoproliferative disorders such as the aforementioned B-cell malignancies. Thus, the skilled person appreciates that the methods exemplified herein with respect to MM are likewise applicable to other B-cell malignancies, especially those characterized by expansion of a B-cell or plasma cell clone as a result of a chromosomal translocation event that yields an immunoglobulin gene fusion.

### Multiple Myeloma

The described methods are particularly useful in the detection and classification of a lymphoproliferative disorder such as multiple myeloma (MM). MM is a plasma cell malignancy characterized by the uncontrolled proliferation of clonal plasma cells within the bone marrow (BM). As used herein, unless otherwise indicated, MM refers to any stage of MM, including precursor conditions, and plasma cell leukemia (PCL), the most aggressive plasma cell disorder.

MM is typically preceded by two precursor conditions: an initial state, called monoclonal gammopathy of undetermined significance (MGUS), followed by smoldering multiple myeloma (SMM). Patients with Multiple Myeloma (MM) and its precursor conditions, Monoclonal Gammopathy of Undetermined Significance (MGUS) and Smoldering Multiple Myeloma (SMM), have variable outcomes depending on the tumor's cytogenetic profile. Many high-risk genomic abnormalities are secondary, meaning that they can be acquired or evolve in a subclone carrying one or more additional abnormalities over time. The methods described herein are useful in the detection of subclones.

Patients with MM and translocation t(4;14), t(14;16), or t(14;20), or gain or amplification of chr1q (Amp1q) and deletion of chr17p (Del17p) have worse prognosis (i.e., so-called high-risk MM) compared to patients with translocation t(11;14) or hyperdiploidy (HRD). Similarly, patients with SMM and t(4;14), Amp1q and deletion of chr13q (Del13q) may be more likely to progress to full-blown MM. Accordingly, the provided methods for determining and classifying a lymphoproliferative disorder such as MM are useful for monitoring disease progression.

Furthermore, the tumor's cytogenetic profile may alter patient management: for example, patients with MM and t(11;14) show superior response to the BCL-2 inhibitor Venetoclax, while patients with high-risk cytogenetic abnormalities may receive a quadruplet (Daratumumab, Lenalidomide, Bortezomib, Dexamethasone) instead of a triplet (Lenalidomide, Bortezomib, Dexamethasone), followed by autologous stem cell transplantation (ASCT) and maintenance with both bortezomib and lenalidomide, compared to potentially skipping ASCT and receiving maintenance with lenalidomide alone (mSMART recommendations). Accordingly, the provided methods for determining and classifying a lymphoproliferative disorder such as MM can be especially useful in identifying a suitable treatment for a patient suffering from the disorder. The provided methods may also be used for monitoring treatment effect.

### Subjects

Subjects to which the provided methods may be applied include those who have been diagnosed with a lymphoproliferative disorder such as a B-cell malignancy (e.g., a B-cell lymphoma, a B-cell leukemia, or a plasma cell dyscrasia such as MM), and/or identified to be at risk from such a disorder (e.g., subjects suffering from MGUS).

A previous diagnosis or identification of risk may be based on one or more of FISH, karyotyping, whole genome sequencing, bone marrow plasma cell (BMPC) number, serum protein immunofixation, serum protein electrophoresis, flow cytometry, lymph node biopsy and immunohistochemistry, complete blood count, or a polymerase chain reaction to detect a tumor-specific mutation. For example, a subject's previous myeloma diagnosis may be based on a combination of FISH and karyotyping (if sufficient numbers of tumor cells are available) as well as serum protein immunofixation and/or serum protein electrophoresis.

Diagnostic information previously obtained from the subject can include the nature, stage and/or cytogenetic features of the underlying lymphoproliferative disease. Such previous analysis may have been carried out on a bone marrow biopsy and a blood sample.

In some cases, the subject has previously been detected to have an elevated level of immune protein or immunoglobulin, such as a hypergammaglobulinemia, and/or paraproteinemia. The nature of the elevated protein (such as determined by serum protein immunofixation) may be used in the provided methods to aid in the selection and/or identification of (pre-) malignant cells, as discussed below.

Diagnostic information previously obtained from a subject may be used, typically alongside similar information from other patients, to train the models and/or algorithms used in the provided methods, as discussed below.

The subject may be or may have been receiving treatment for a lymphoproliferative disorder such as a B-cell malignancy, prior to being diagnosed or classified with a method provided herein. The treatment may have been selected based on previous classification, such as cytogenetic classification, of the lymphoproliferative disorder. The subject may be or may have been in remission.

Subjects may or may not have undergone treatment for their lymphoproliferative disorder. For example, if the subject is suffering from a B-cell malignancy in a premalignant and/or presymptomatic state, such as MGUS, the subject typically is not undergoing treatment.

As demonstrated in the examples provided below, the provided methods (including the computer-implemented methods described herein) often detect lymphoid clones that have not been detected clinically (e.g., using FISH or other standard diagnostic methods). Thus, in some aspects, the provided methods may be used for screening subjects, such as subjects that belong to an at-risk population, including subjects above a certain age (e.g., older than 50 or older than 60 years of age).

The above information concerning the subject can equally apply to the subjects whose information is used for the preparation and/or training of the statistical models and/or machine learning algorithms described below.

### Sample Preparation and Enrichment

There is provided a method for detecting or classifying a lymphoproliferative disorder such as a B-cell lymphoma, a B-cell leukemia, or a plasma cell dyscrasia in a subject, as well as methods of screening subjects for the presence of a lymphoproliferative disorder such as a B-cell lymphoma, a B-cell leukemia, or a plasma cell dyscrasia. Such methods are performed on a sample obtained from a subject.

### Samples

A bone marrow biopsy or other invasive biopsy (such as lymph node biopsies) is usually required to, e.g., diagnose, classify and/or monitor lymphoproliferative disorders such as B-cell lymphomas, B-cell leukemias, or plasma cell dyscrasias (e.g., MM). Methods provided herein can be used with such biopsy samples, and the improved sensitivity and increased information afforded by these methods can be useful in increasing the efficacy of classification of a lymphoproliferative disorder.

A major advantage of the provided methods is that they can additionally be used with liquid biopsies such as lymph liquid samples and blood samples. Given their effectiveness in the classification of lymphoproliferative disorders such as B-cell lymphomas, B-cell leukemias, and plasma cell dyscrasias (e.g., MM) even with very small number of tumor cells (e.g., 50 cells or fewer, 20 cells or fewer, or 10 cells or fewer), they may avoid the need for an invasive biopsy altogether. Lymph liquid or blood samples can be obtained with minimal invasiveness, especially in the case of peripheral blood, and can be taken frequently in order to monitor disease progression, while minimizing the risks and discomfort associated with invasive biopsies. Methods such as those provided herein may have previously been applied to one or more samples from a subject, especially where the aim is to monitor disease progression. Accordingly, the sample may be a blood sample, a lymph node tissue obtained by biopsy, a bone marrow tissue obtained by biopsy, or a tumor tissue obtained by biopsy.

In particular, the sample may be a blood sample. Blood samples frequently contain circulating tumor cells (CTCs) derived from the lesion underlying a lymphoproliferative disorder that typically originates in the bone marrow, such as a B-cell malignancy including a B-cell lymphoma, a B-cell leukemia, or a plasma cell dyscrasia. Where clonal diversity is present, a blood biopsy allows a sample of multiple, and potentially all, clones present in the bone marrow instead of sampling only one site of bone marrow, thus providing a more complete profile of a tumor. This is particularly important when considering the genetic evolution a tumor undergoes over time or post-treatment, when newly acquired or selected—for alterations tend to be present in only a portion of malignant cells (i.e., subclonal alterations) and therefore their frequency (i.e., subclone size) may vary from tumor niche to tumor niche across the body. Therefore, CTC-based profiling may provide a more representative snapshot of the tumor's genetic architecture across all tumor niches in the body.

### Enrichment

Samples (in particular blood samples, but also bone marrow samples) generally require enriching for the type of cells likely to contain the tumoral (i.e., malignant or pre-malignant) cells of the lymphoproliferative disorder (e.g., the B-cell malignancy), and/or for the tumoral cells themselves. For example, starting with a blood sample, the cells in the sample are typically enriched for mononuclear cells and/or subsets thereof, such as peripheral blood mononuclear cells (PMBCs). Enrichment may comprise positive and/or negative selection, e.g., using one or more cell surface markers.

For example, enriching the sample may comprise using erythrocyte aggregation/sedimentation; fluorescence-activated cell sorting (FACS), immunofluorescence-based techniques; and/or magnetic bead enrichment.

An enrichment procedure may include multiple steps. For example, blood samples are typically processed to isolate PBMCs by a process including the aggregation and sedimentation of erythrocytes, followed by labelling and removal of non-PBMCs, e.g., using one or more antigen-binding moieties such as antibodies that target granulocytes and/or residual erythrocytes. Similar methods may be used to enrich for bone marrow mononuclear cells.

For particular underlying disorders, it may be useful for the sample to be enriched, or further enriched, for a specific cell type, such as B cells, plasma cells or T cells. In disorders such as certain B-cell malignancies including MM, the samples are typically enriched for plasma cells. In some conditions, it may be possible to enrich for tumoral cells directly, for example by selecting for cell surface markers characteristic of the tumoral cells (i.e., tumor cell surface markers). In some instances, a DAPI stain may be used to select for and/or detect nucleated cells.

Specific methods for enrichment are known in the art, and can include immunophenotype-based enrichment methods, immunomagnetic and/or immunofluorescence imaging technology, fluorescence activated cell sorting (FACS, e.g., high-sensitivity FACS).

General methods can include those wherein antigen-binding moieties (e.g., antibodies) are employed that specifically bind to one or more antigens expressed on the cell surface of the cells for selection or removal. The one or more antigens may be CD138 (which is expressed on plasma cells), CD19 (which is expressed on B cells), or CD3 (which is expressed on T cells).

For example, the immunophenotypes CD138+ and/or CD38+ can be used to select for plasma cells. The immunophenotype CD3− can be used to exclude T cells. Suitable immunophenotypes are known in the art for many lymphoproliferative disorders, including B-cell lymphomas, B-cell leukemias, and plasma cell dyscrasias (e.g., MM). Non-limiting examples include the gain of CD56 and CD117 and loss of CD19, CD45, and CD27 in myeloma cells. Thus, one or more of these immunophenotypes (CD56+, CD117+, CD19−, CD45−, and CD27−) can be used in addition to CD138 to select for a cell of a subject that suffers from, or is suspected to suffer from, a lymphoproliferative disorder such as MM.

Other non-limiting examples include the immunophenotypes CD20^{dim}, CD5+, CD23+, CD43+ and LEF1+ in chronic lymphocytic leukemia cells. Thus, one or more of these immunophenotypes (CD20^{dim}, CD5+, CD23+, CD43+ and LEF1+) can be used to select for a cell of a subject that suffers from, or is suspected to suffer from, a lymphoproliferative disorder such as chronic lymphocytic leukemia.

Cell surface markers characteristic of tumoral cells found in other lymphoproliferative disorders such as B-cell lymphomas, other B-cell leukemias, and other types of plasma cell dyscrasia are well known in the art.

At the expression level (using single-cell RNA sequencing data), one or more of these immunophenotypic markers can also be used to determine whether a cell is malignant (using, e.g., a tumor cell profiling algorithm as described herein).

While the exemplified methods provided herein are focused on the isolation of plasma cells for the diagnosis or classification of MM, it is understood that corresponding enrichment steps can be used to enrich for pre-malignant or malignant cells of lymphoproliferative disorders other than myelomas, such as B-cell lymphomas, B-cell leukemias, and other types of plasma cell dyscrasia. Depending on the type of the lymphoproliferative disorder (e.g., a B cell lymphoma or a T cell lymphoma), different cells may be selected, isolated or enriched (e.g., B cells or T cells). Accordingly, the enrichment step may select for T cells B cells. In particular, the skilled person appreciates that the methods exemplified herein with respect to MM are applicable to other B-cell malignancies, especially those characterized by expansion of a B-cell or plasma cell clone as a result of a chromosomal translocation event that yields an immunoglobulin gene fusion.

### Single-Cell RNA Sequencing

Following enrichment for or isolation of a particular cell type, single-cell RNA sequencing is carried out on the sample. Any suitable method known in the art may be used for such sequencing.

Typically, methods for single-cell RNA sequencing involve dilution of the cells in the sample, followed by microfluidic partitioning, encapsulation, and recovery of single cells. RNA is then isolated from single cells and reverse transcribed into cDNA. Sequencing libraries are prepared by barcoding the cDNAs originating from individual cells and sequencing the barcoded cDNAs.

For example, samples, gel bead-in-emulsions (GEMs), and partitioning oil may be loaded into a Next GEM Chip K microfluidic device and placed in a Chromium Controller instrument (10× Genomics) for single-cell encapsulation and recovery. Barcoding, reverse transcription of the RNA, cDNA amplification, and library construction can be completed using commercially available kits, such as the Chromium Next GEM Single Cell 5′ Reagent Kit v2 (Dual Index) and Library Construction Kit (10× Genomics), according to the manufacturer's instructions.

The generated sequencing information can include data for immunoglobulin and/or T cell receptor expression. The identification of clonal cell populations (e.g., clonal myeloma or lymphoma cell populations) can be based on this information. Accordingly, single-cell B cell receptor (BCR) and/or T cell receptor (TCR) sequencing data may be obtained, e.g., by preparing corresponding libraries. For single-cell BCR sequencing V(D)J cDNAs may be amplified prior to library construction.

An exemplary method comprises generating cDNA from the isolated RNA of individual cells, e.g., as described above, and subjecting the cDNA to V(D)J amplification, e.g., using commercially available kits, such as the Chromium Single Cell Human BCR Amplification kit and Library Construction kit (10× Genomics), according to the manufacturer's instructions.

Typically, sequencing data are subjected to methods to improve the quality of data. Suitable methods can be used to remove ambient RNA, low-quality cells (such as those with high mitochondrial gene expression), and to remove multiplets. Gene expression matrices can be generated, e.g., using CellRanger count provided by 10× Genomics (Zheng et al. Nat Commun (2017) 8:14049). Ambient RNA can be removed, e.g., using CellBender (v0.2.0), by applying a target false positive rate cut-off of 0.01. Single-cell RNA sequencing data may be characterized as “low-quality cells”, e.g., if the data comprise >15% mitochondrial gene expression, and either <200 detected genes, >5,000 detected genes, <400 UMIs, or >50,000 UMIs. Scrublet (v0.2.3) (Wolock et al. Cell Syst (2019) 8(4):281-291 Germain et al. F1000Res (2022), 10:979), or scds (1.10.0) (Bais & Kostka Bioinformatics (2020), 36:1150-1158) can be used to calculate multiplet scores.

### Tumor Cell Profiling Algorithm

In order to detect or classify a lymphoproliferative disorder such as a B-cell lymphoma, a B-cell leukemia, or a plasma cell dyscrasia (e.g., MM) in a subject, the provided methods apply a tumor cell profiling algorithm to single-cell RNA sequencing data including, e.g., single-cell BCR V(D)J sequencing data. The algorithm identifies (i) cells expressing one or more lineage markers of the cell type comprising the tumoral cells; (ii) identifies among the cells expressing the one or more lineage markers one or more expanded clonal cell populations, e.g., based on the BCR V(D)J sequencing data; (iii) categorizes each of the one or more expanded clonal cell populations as malignant or pre-malignant if expression of a pre-determined set of oncogenes and tumor surface markers specific to lymphoproliferative disorder, e.g., the B-cell lymphoma, the B-cell leukemia, or the plasma cell dyscrasia, is detected; and optionally, but advantageously (iv) determines the (median) expression of translocation partner genes and/or the presence of one or more copy number variants (CNVs) in the one or more malignant or pre-malignant tumor cell population(s).

### Identification of Cells Using Lineage Markers

The single-cell RNA sequencing data may be analyzed by the tumor cell profiling algorithm to identify single-cell sequencing data (e.g., cell barcodes) corresponding to cells of interest, typically prior to identifying expanded clonal cell populations. Cells of interest may be identified based on their expression of one or more lineage markers.

For example, where the underlying lymphoproliferative disorder is, or is thought to be, MM, single-cell RNA sequencing data relating to plasma cells are identified based on the expression of plasma cell markers such as SDC1 (encoding CD138), CD38, XBP1, PRDM1, IRF4, and TNFRSF17 (encoding BCMA).

In other instances, e.g., where the lymphoproliferative disorder is a T cell lymphoma, one or more markers of T cell lineage are identified.

Similarly, where the lymphoproliferative disorder is a B cell lymphoma, one or more markers of B cell lineage may be identified.

Single-cell RNA sequencing data (e.g., cell barcodes) that are determined to correspond to cell of interest are considered for downstream analysis.

### Clone Identification and Analysis

Lymphoproliferative disorders such as B-cell lymphomas, B-cell leukemias, and plasma cell dyscrasias originate from the uncontrolled expansion of lymphocytes (e.g., B-cells or plasma cells), usually originating from a single clone, and frequently following a cytogenetic change such as a translocation, or other chromosomal abnormality such as a focal or arm-level deletion, focal or arm-level gain or amplification, focal or arm-level loss of heterozygosity, monosomy, trisomy, tetrasomy, pentasomy, whole-genome doubling, or hyperdiploidy.

For example, the tumor cell profiling algorithm can identify lymphocyte clone populations by the nature of their expressed immunoglobulin, that is, B cell receptor or T cell receptor. Due to how these receptors vary between clones, as a result of the recombination of V (Variable), D (Diversity), J (Joining), and C (Constant) genes, analysis of V(D)J-C sequences provides a convenient natural barcode for identifying clonal cell populations. In the event of a B-cell malignancy, the tumor cell profiling algorithm analyzes the BCR V(D)J sequencing data to identify one or more expanded clonal cell populations. For conciseness, BCR V(D)J sequencing data are also referred to as scBCRseq data herein.

For example, V(D)J contigs can be detected using, e.g., CellRanger (v6.0.1) (Zheng et al., supra). V(D)J contigs may be processed to identify a single heavy and light chain per cell barcode, e.g., based on Unique Molecular Identifier (UMI) support. Occasionally, clonotypes with only a heavy or light chain may be identified. Such single-chain clonotypes may be collapsed with the matching two-chain clonotype that has the highest frequency.

Expanded clonal cell populations (i.e., populations of proliferating lymphocytes) are likely candidates for pre-malignant or malignant cell populations. Detection of expanded clonal populations may be achieved by any suitable method, including setting a frequency cut-off, or by assessing relative frequency.

For example, a clonal cell population may be identified as expanded if the clonal cell population is at least 1.2-fold, at least 1.5-fold, or at least 2-fold the size of any other clonal cell population detected in the sample. In particular, a clonal cell population may be identified as expanded if the clonal cell population is at least 1.2-fold, at least 1.5-fold, or at least 2-fold the size of the median, mode or mean size of all clonal cell populations detected in the sample.

Another suitable method comprises sorting the clonal cells detected in the sample by frequency (cell count) and selecting a clonal population of cells as expanded if the clonal cells have a frequency at least 50% higher, at least 100% higher, at least 150% higher, at least 200% higher, or at least 300% higher than the next most frequent clonal cells in the sample. In particular, a clonal cell population may be identified as expanded if the clonal cells have a frequency at least 200% higher than the most frequent clonal cells in the sample.

An expanded clonal cell population may be identified if it comprises at least 5 cells, at least 10 cells, or at least 20 cells. In particular, an expanded clonal cell population may be identified if it comprises at least 20 cells.

### Tumor Cell Identification

Expanded clonal populations may not necessarily represent a tumoral cell of an underlying lymphoproliferative disorder such as a B-cell lymphoma, a B-cell leukemia, or a plasma cell dyscrasia (e.g., MM). For example, expanded populations may arise for a number of reasons even in healthy subjects, such as an immune response following vaccination or exposure to a pathogen. Therefore, further analysis is carried out by the tumor cell profiling algorithm to identify whether a given expanded clonal population is pre-malignant or malignant.

This identification is typically achieved by determining the presence of a pre-determined set of malignancy markers, e.g., oncogenes and tumor surface markers specific to a lymphoproliferative disorder such as a B-cell lymphoma, a B-cell leukemia, or a plasma cell dyscrasia (e.g., MM), in the single-RNA sequencing data representing the expanded clonal cell population. The malignancy markers may be common to multiple proliferative disorders, e.g., to multiple lymphoproliferative disorders. More typically, however, the malignancy markers are specific to the lymphoproliferative disorder (e.g., the B-cell malignancy) that is detected or classified by the disclosed method.

For example, key oncogenes that may be used for the detection of MM (as well as precursor conditions such as MGUS and SMN) include CCND1, MAF, and WHSC1 (which is also known as MMSETINSD2). Key oncogenes may include one or more of (e.g., two, three, four five, six, seven, eight, nine, ten, or all of) CCND1, CCND2, CCND3, MAF, MAFA, MAFB, ITGB7, NSD2, FGFR3, LAMP5, and FRZB. These include well-characterized chromosome translocation partner genes and genes that are commonly upregulated in patients with MM.

CCND1 expression is upregulated in other lymphoproliferative disorders in addition to MM. For example, CCND1 expression is upregulated in mantel cell lymphoma (Puente et al. Blood (2018), 131 (21): 2283-2296) and in hairy cell leukemia (Boer et al. Annals of oncology (1996), 7 (3): 251-255).

One or more immunophenotypic markers (e.g., one or more tumor surface antigens) may also be used as one or more malignancy markers. For example, immunophenotypic markers (e.g., tumor surface markers) that may be used to identify a pre-malignant or malignant plasma cell include one or more of NCAM1 (CD56), CD19, and PTPRC (CD45). Other suitable immunophenotypic markers include CD27, CD117 (KIT), and CD28.

It is understood that commonly known and validated sets of malignancy markers such as key oncogenes and tumor surface markers for specific lymphoproliferative diseases such as B-cell lymphomas, B-cell leukemias, and plasma cell dyscrasias may be used in implementing the methods disclosed herein. Suitable sets of malignancy markers are known for the various lymphoproliferative diseases disclosed herein (including the aforementioned B-cell malignancies), as illustrated for MM in the examples.

Because a single tumor of a B-cell malignancy can present with variants of a malignant clonotype (for example, a light chain only variant of the main clonotype), consensus clonotypes can be determined for each tumor by sorting malignant clonotypes based on the number of tumor cells using them and selecting the top clonotype. For each consensus clonotype, the Levenshtein distance between each clonotype detected in the sample and the consensus clonotype can be computed on the CDR3 amino acid sequence and normalized by the length of the longest of the two sequences. This provides a measure of the similarity of the clonotypes in the tumor and the consensus clonotype for that tumor. A clonotype can be excluded from the normal B-cell or plasma cell compartment if the similarity between a reference sequence (such as the CDR3 amino acid sequence) for a clonotype and consensus clonotype exceeds a threshold. For example, clonotypes with at least 90% similarity to the consensus clonotype can be considered variants of the malignant clonotype and annotated as malignant, whereas clonotypes with at least 60% but less than 90% similarity can be considered suspicious and excluded from the normal B-cell or plasma cell compartment.

A method disclosed herein may also use information previously obtained from the same subject to identify malignant cells, e.g., by comparing expression information for a clonal cell population with a previously reported value for the subject. For example, the oncogene markers discussed above may be chosen by their matching those expected from the subject's previously determined cytogenetic profile (such as that detected by FISH, genome sequencing, or by previous application of the presently described methods). Similarly, the isotype of the expressed immunoglobulin can be compared to that previously reported clinically for the subject, for example where the previous report was determined by serum protein immunofixation, serum protein electrophoresis, immunohistochemistry, in situ hybridization, flow cytometry, or previous application of present methods.

### Expression of Translocation Partner Genes

The method may further comprise determining the expression of translocation partner genes for the one or more malignant or pre-malignant tumor cell population(s). Translocation partner genes are genes whose expression is associated with a chromosomal translocation. Translocation partner genes may vary between lymphoproliferative disorders such as B-cell lymphomas, B-cell leukemias, and plasma cell dyscrasias (e.g., MM). The expression of translocation partner genes is a useful metric because it can be used as a proxy to infer the presence of translocations, as will be explained in more detail below.

Determining the expression of translocation partner genes comprises scoring each cell in a malignant or pre-malignant tumor cell population for the expression of one or more key gene signature(s) (such as CD-1, CD-2, MS, MF, and HY for MM) and the expression of key oncogenes (e.g., CCND1, CCND2, CCND3, NSD2, FGFR3, MAF, MAFA, MAFB, and ITGB7 for MM). The key gene signatures and key oncogenes are referred to collectively as translocation partner genes. Other gene signatures and oncogenes may be considered where the lymphoproliferative disorder is not MM.

The scoring is based on the single-cell RNA sequencing data and may use Scanpy's (v1.8.2) score_genes function. The gene signature scores may be normalized, such as by min-max normalization. The oncogene scores may be subjected to a log1p transformation. Other known methods of scoring gene expression can be used.

For each tumor cell population, a median signature score for the expression of each key gene signature can then be calculated using the (normalized) scores, and a mean score for the expression of each key oncogene can be calculated using the (transformed) scores. The median signature scores represent the expression of the key gene signatures for that tumor cell population. The mean oncogene scores represent the expression of the key oncogenes for that tumor cell population.

A given gene, particularly a gene expressed at low levels such as a transcription factor that is upregulated as the result of a chromosomal translocation at an immunoglobulin locus, may not be detected in all cells that express the gene due to drop-outs that often occur with droplet-based single-cell RNA sequencing data. Mean gene expression at the tumor level can be used to overcome drop-out phenomena.

### Copy Number Variation

The method may further comprise inferring copy number variants (CNVs) from the single-cell RNA sequencing data obtained for the one or more malignant or pre-malignant clonal cell population(s).

Existing methods for the inference of CNVs can be used in the present methods. CNVs are particularly useful for the cytogenic profiling of expanded clonal population of pre-malignant or malignant cells, e.g., to detect chromosomal abnormalities such as amplifications and deletions, as discussed further below.

Suitable methods useful in determining CNVs from single-cell RNA sequencing data are described, e.g., in Gao, T., et al. Nat Biotechnol (2023) 41:417-426). Gao et al. describe a computational approach that integrates haplotype information obtained from population-based phasing with allele and expression signals to enhance detection of CNVs from single-cell RNA sequencing data. Such approaches can also be useful in identifying pre-malignant or malignant cells, and in determining subclonal complexity by allele-specific alterations.

Accordingly, the computational methods disclosed herein may integrate expression data including allelic data from pre-malignant or malignant cells to detect characteristic CNVs in single-cell RNA sequencing data in order to enhance the identification of data sets deriving from tumor cells (e.g., CTCs) and characterize them.

Genome aberrations may be characterized by determining an allelic imbalance or changes in expression magnitudes. An allelic imbalance reflects relative copy numbers of two homologous chromosomes. Changes in expression magnitudes reflect the total chromosomal dosage. A joint Hidden Markov model (HMM) based on a generative statical framework can be used to combine the expected expression shifts and allelic ratio corresponding to a potential copy number configuration. To increase robustness, gene expression can be modeled as integer read counts using a discrete Poisson Lognormal mixture distribution. In addition, excess variance in the allele frequency (e.g., due to allele-specific detection or transcriptional bursts) can be accounted for by using a beta-binomial distribution. The expression and allele signals in single cells can be integrated via such a generative statical framework to produce probabilistic estimates of copy numbers in single cells. The balanced regions are then clustered based on the expression shifts relative to a normal reference (e.g., derived from normal plasma cells, B-cells, or T-cells depending on the lymphoproliferative disorder), and the cluster with the lowest average expression fold change is then designated as diploid regions.

Allelic data can be collected from the mononuclear cells (e.g., expanded clones of plasma cells, B cells, or T cells) from which single-cell RNA sequencing data has been obtained. These data may be processed to ensure that the frequency of CNVs and particularly, clonal deletions, does not negatively impact genotyping and phasing in samples with high tumor purity. An expression reference, for example, collected from healthy donor cells of the same type, can be used to correct for cell type-specific expression patterns, such as immunoglobulin expression, which otherwise result in artifactual CNV inference on particular chromosomes. For example, CNVs can be determined using Numbat (v1.1.0), as described herein.

For instance, where the lymphoproliferative disease is MM, allelic data may be collected from plasma cells (including malignant and normal ones) and B cells to ensure that the frequency of CNVs and particularly, clonal deletions, do not negatively impact genotyping and phasing in samples with high tumor purity. The allelic data obtained from normal plasma cells provides a subject-specific baseline against which CNVs in malignant cells can be identified. Furthermore, a custom panel of healthy donor plasma cells can be used as expression reference to correct for cell type-specific expression patterns, such as immunoglobulin expression, which otherwise result in artifactual CNV inference on chromosomes 2, 14, and 22. In other words, expression data for healthy donor plasma cells provides a baseline to which expression patterns of pre-malignant or malignant cells can be compared.

CNVs can be inferred on tumor cells only to increase detection sensitivity. Samples with large number of tumor cells (e.g., >15,000 tumor cells) can be downsampled prior to running Numbat for efficiency. Numbat can be run with max_iter=2, tau=0.1, min_cell=10, to further increase detection sensitivity.

An expression-based probability that a given CNV exists in a cell can be determined for each cell in a malignant or pre-malignant tumor cell population using the single-cell RNA sequencing data. In addition, an allele-based probability that the CNV exists in the cell can be determined for each cell in the malignant or pre-malignant clonal cell population using the single-cell RNA sequencing data. Joint CNV probabilities can then be calculated using both the expression-based probabilities and the allele-based probabilities for each CNV and for each cell in a malignant or pre-malignant tumor cell population.

CNVs are typically inferred on a combined list of tumor cell barcodes (representing an expanded clonal cell population) to increase sensitivity. Inference can be done by combining single-RNA sequencing data from multiple samples (e.g., bone marrow and peripheral blood samples).

### Identification of Subclones

Also provided is a computer-implemented method for identifying a copy number variant(CNV) profile of one or more subclones in malignant or pre-malignant tumor cell populations in a subject suffering from lymphoproliferative disorder such as a B-cell malignancy (e.g., a B-cell lymphoma, a B-cell leukemia, or a plasma cell dyscrasia such as MM), wherein the method comprises:
(a) obtaining single-cell RNA sequencing data (e.g., including single-cell B cell receptor (BCR) V(D)J sequencing data) for a population of cells, wherein the population of cells was obtained from a sample obtained from the subject by enriching the sample for a cell type comprising tumoral cells using a cell surface marker;
(b) applying a tumor cell profiling algorithm to the single-cell RNA sequencing data; wherein the algorithm:
   i. identifies cells expressing one or more lineage markers of the cell type comprising the tumoral cells;
   ii. identifies among the cells expressing the one or more lineage markers one or more expanded clonal cell populations (e.g., based on the BCR V(D)J sequencing data);
   iii. categorizes each of the one or more expanded clonal cell populations as malignant or pre-malignant if expression of one or more malignancy markers (e.g., a pre-determined set of oncogenes and tumor surface markers specific to the B-cell malignancy) is detected;
   iv. infers joint CNV probabilities based on expression and allelic data of the malignant or pre-malignant tumor cell population(s);
   V. clusters cells within each malignant or pre-malignant tumor cell population based on the joint CNV probabilities to identify subclones of cells within said cell population;
   vi. identifies the CNV profile a subclone by: (1) calculating a median joint CNV probability for each CNV of the cells in each potential subclone and (2) assessing, for each CNV, whether the median joint CNV probability is above a predetermined threshold value.

In addition to the inference of CNV information as described above, CNV profiles for subclones can be identified by the algorithm. For a given tumor cell population, the cells can be clustered in the CNV feature space based on the joint CNV probabilities calculated above. Any known clustering method can be used, such as hierarchical clustering, density-based clustering, or subspace clustering. In some examples, clustering can be implemented by agglomerative hierarchical clustering using Euclidean distances. Each cluster is a potential subclone of the tumor.

The CNV profile of a potential sub-clone can be identified by calculating a median joint CNV probability for each CNV based on the joint CNV probabilities of the cells in a potential subclone and assessing, for each CNV, whether the median joint CNV probability is above a predetermined threshold value. For example, the threshold value may be 0.3, 0.5, 0.55, 0.6, 0.65 or 0.8. The predetermined threshold may be any value in the range 0.3-0.8. The predetermined threshold may be any value in the range 0.5-0.8. The threshold may be greater than 0.8.

Identifying the CNV profile of a subclone may comprise identifying a CNV as being part of the subclone's CNV profile if that CNV has a median joint CNV probability greater than the predetermined threshold.

The CNV profile of a subclone may be used for phylogenetic tree inference via maximum parsimony analysis

The tumor cell profiling algorithm can further determine the size of each subclone by dividing the number of cells in a subclone by the total number of cells within the malignant or pre-malignant tumor cell population comprising the subclone. The identification of subclones and/or obtaining information on the size of subclone can be used for prognosis and/or guide the selection of an appropriate treatment or therapeutic regimen.

As demonstrated in the examples provided below, the provided methods are capable of determining subclone size of malignant clonal cell populations more accurately than WGS. Accordingly, the provided methods described here can be used to determine the subclone size of malignant clonal cell populations.

### Exemplary Tumor Profiling Algorithm

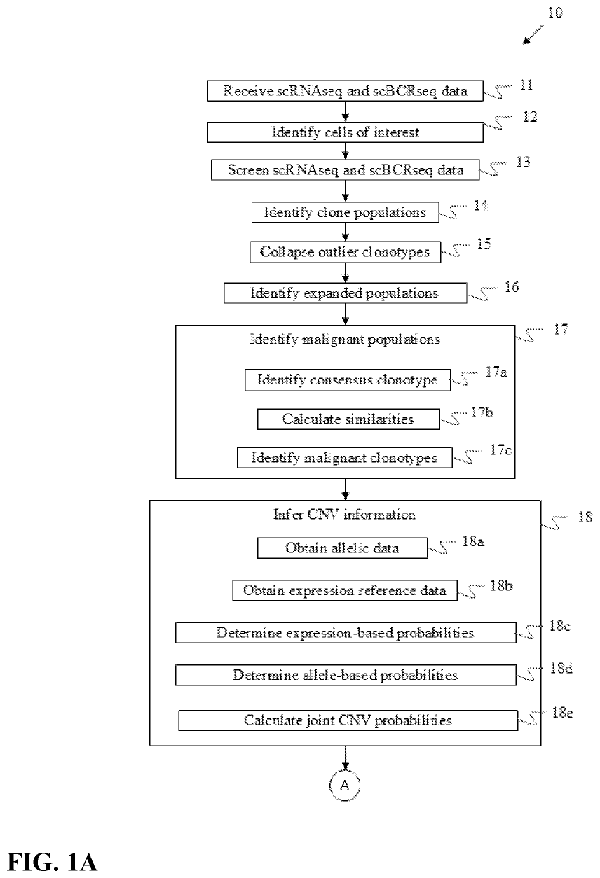

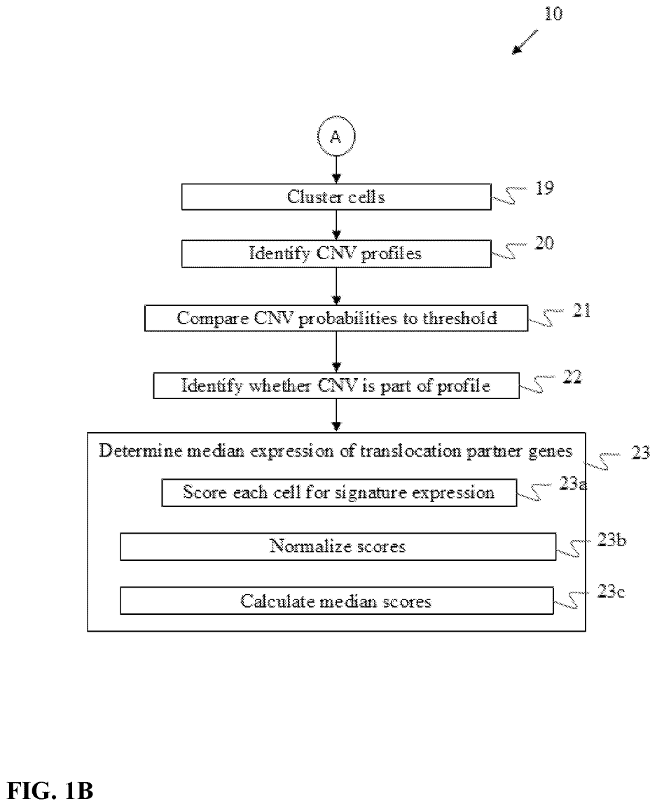

An example of the computer implemented method 10 performed by the tumor cell profiling algorithm to detect or classify a B-cell malignancy such as a B-cell lymphoma, a B-cell leukemia, or a plasma cell dyscrasia (e.g., MM) will now be described with reference to FIGS. 1A and 1B.

Starting with FIG. 1A, the tumor cell profiling algorithm receives 11 as an input single-cell RNA (scRNAseq) sequencing data and single-cell BCR V(D)J sequencing (scBCRseq) data. In some examples, the scRNAseq data and scBCRseq data may consist of data for cells of interest (i.e., B-cells or plasma cells). However, in some examples the data may include data for other types of cells that are not of interest. Accordingly, in some examples, cells of interest are identified 12 based on the scRNAseq data. As explained above, this can be achieved by analyzing the scRNAseq data for linage markers of the cells of interest. The scRNAseq data is screened 13 to filter out scRNAseq data for cells that are not of interest.

Next, clone populations are identified 14. As explained above, this can be achieved by analyzing the scBCRseq data. Outlier clonotypes (for example, clonotypes with only a heavy or light chain) are collapsed 15 with (that is, grouped with) the matching two-chain clonotype that has the highest frequency.

Next, expanded clonal cell populations are identified 16. As explained above, an expanded clonal cell population is a clonal cell population having a size greater than a threshold. In the present example, this threshold is 2-fold the size of any other clonal cell population detected in the sample. In other examples, expanded clonal cell populations may be identified based on the frequency at which they occur or their size relative to a mean, median or modal size of the clonal cell populations. For example, a clonal cell population may be identified as expanded if the clonal cells have a frequency at least 200% higher than the next most frequent clonal cells in the sample. In other examples, a clonal cell population may be identified as expanded if it contains more than a threshold number of cells. For example, a clonal cell population containing more than 5 cells, more than 10 cells or more than 20 cells can be identified as an expanded clonal cell population.

The algorithm identifies 17 each expanded clonal population as pre-malignant or malignant, or as normal. As explained above, the identifying 17 can be based on determining the presence of a pre-determined set of malignancy markers (e.g., oncogenes and tumor surface markers specific to the B-cell malignancy) in the scRNAseq data representing the expanded clonal cell population. In some examples, a tumor may present with more than one variant of a malignant clonotype. For example, a light chain only variant of the main clonotype. Depending on the tumor surface markers used to identify pre-malignant and malignant cells, the light-chain only variant may lack a malignancy marker despite being otherwise very similar or identical to the malignant clonotype. To avoid this variant being incorrectly classified as belonging to the normal plasma cell compartment, a consensus clonotype is identified 17a for the tumor. The consensus clonotype is the largest clonotype for the tumor (that is, the clonotype having the largest number of cells). A similarity between the consensus clonotype and each of the other clonotypes for the tumor is calculated 17b. For example, normalized Levenshtein distances may be calculated to compare the consensus clonotype and the other clonotypes for the tumor. Clonotypes having at least 90% similarity to the consensus clonotype are considered variants of the malignant clonotype and are identified 17c as malignant. Clonotypes having between 90% and 60% similarity are considered suspicious and are excluded from the normal plasma cell compartment.

As explained above, in other examples pre-malignant or malignant populations may additionally or alternatively be identified based on a comparison of expression information for a clonal cell population with a previously reported value for the subject.

CNV information is inferred 18 for the malignant or pre-malignant clonal cell populations. Inferring CNV information comprises: obtaining 18a allelic data for malignant and normal B-cells and/or plasma cells (depending on the B-cell malignancy) obtained from the subject; obtaining 18b expression reference data from a sample of normal B-cells or plasma cells (e.g., from a group of healthy donors); determining 18c, for each cell, expression-based probabilities that each CNV exists in that cell based on the scRNAseq data for that cell and the expression reference data; determining 18d, for each cell, allele-based probabilities that each CNV exists in that cell based on the scRNAseq data for that cell and the allelic reference data; and calculating 18e a joint CNV probability for each CNV for each cell, based on the expression-based probability and the allele-based probability. The joint CNV probability may be calculated as the product of the expression-based probability and the allele-based probability.

Turning to FIG. 1B, which continues the method 10, cells within each malignant or pre-malignant tumor cell population are clustered 19 in the joint CNV probability feature space. In the present example agglomerative hierarchical clustering using Euclidean distances is used; however, other known clustering methods can be used instead. Each cluster represents a potential sub-clone of the tumor.

A CNV profile for each potential sub-clone is identified 20 by calculating a median joint CNV probability for each CNV of the cells in each potential subclone. The median joint CNV probabilities are each compared 21 to a predetermined threshold value. In the present example, the threshold value is 0.5; however, other values may be used in other examples. For each potential sub-clone, a CNV is identified 22 as being part of the subclone's CNV profile if the median joint CNV probability for that CNV is greater than the predetermined threshold.

The algorithm determines 23 the median expression of translocation partner genes in the malignant or pre-malignant tumor cell populations. More specifically, in the present example the algorithm scores 23a each cell in a malignant or pre-malignant tumor cell population for the expression of key gene signatures, based on the scRNAseq data. The scores are normalized 23b using min-max normalization. A median signature score for each key gene signature is calculated 23c for each malignant or pre-malignant tumor cell population.

In some examples, the algorithm may perform only one of inferring 18 CNV information and determining 23 the median expression of translocation partner genes. In some examples, the algorithm may infer 18 CNV information but may not proceed through steps 19-22 of the method. In some examples, the algorithm may determine 23 of the median expression of translocation partner genes before, or in parallel with, the inference 18 of CNV information.

Steps 12, 13, 15, 17a-17c and 18-23 are optional, and one or any combination of these steps may not be required in some instances. For example, the algorithm may not perform steps 18-23 in instances in which the use of the algorithm is limited to identifying malignant populations. The algorithm may not perform steps 12 and 13 in instances in which the scRNAseq data and scBCRseq data obtained by the algorithm has been pre-processed to filter out data for cells not of interest. The algorithm may not perform step 15 in instances in which the sample is known not to contain any outlier clonotypes or in which the scRNAseq data and scBCRseq data has been pre-processed to filter out any outlier clonotypes, or to reduce the computational burden of the algorithm. In some instances, outlier clonotypes may simply be disregarded or ignored by the algorithm if they are identified. Similarly, the algorithm may not perform steps 17a-17c in instances in which the scRNAseq data and scBCRseq data has been pre-processed to filter out variant clonotypes, or to reduce the computational burden of the algorithm. In some instances, variant clonotypes may be disregarded or ignored by the algorithm if they are identified. In some instances, the algorithm may perform only steps 11, 14, 16 and 17.

### Disorder Classification

Having identified data corresponding to malignant or pre-malignant tumor cell populations within the collected data, the sequencing data can be used to classify the underlying lymphoproliferative disorder, e.g., using cytogenetic profiling based on CNVs as described above. The cytogenetic profile may comprise one or more translocations and/or one or more copy number abnormalities, such as hyperdiploidy. Such classifications may relate to chromosomal translocations characteristic of subcategories of a lymphoproliferative disorder, in particular subcategories of B-cell malignancies such as B-cell lymphomas, B-cell leukemias, and plasma cell dyscrasias.

Chromosomal translocations are commonly detected in hematological malignancies such as lymphoproliferative disorders, especially B-cell malignancies. For example, in MM and B-cell lymphomas, chromosomal translocations involving immunoglobulin loci are common transforming events. In MM, chromosomal translocations include t(11;14), t(4;14), t(14;16), t(14;20), t(8;14), and t(6;14). The primary translocation in follicular lymphoma is t(14;18)(q32;q21), the primary translocation in mantel cell lymphoma is t(11;14)(q13;q32), and the primary translocation in Burkitt lymphoma is t(8;14)(q24;q32) (Grau et al., supra).

Other cytogenetic events such as mutations in tumor suppressor genes, genomic amplification, and translocations not involving immunoglobulin loci have also been implicated in these conditions. This is discussed, for example, in Küppers, R. (2005) Nat Rev Cancer 5, 251-262. Classifications may accordingly also include other chromosomal abnormalities such as aneuploidy, monosomy, chromosomal amplification (e.g., trisomy, tetrasomy, pentasomy), focal or arm-level gains, loss of heterozygosity, chromosomal deletion (e.g. focal or arm-level deletions), and hyperdiploidy. Recurrent trisomies may involve odd-numbered chromosomes with the exception of chromosomes 1, 13, and 17, which more commonly show Del1p, Amp1q, Del13q and Del17p.

Accordingly, the method may further comprise a step of analyzing the one or more malignant or pre-malignant tumor cell population(s) using a statistical model or machine learning algorithm to classify the lymphoproliferative disorder, e.g., the B-cell malignancy. Classifying the disorder may comprise generating, by the statistical model or machine learning algorithm, a cytogenetic classification of the disorder based on the single-cell RNA sequencing data of the one or more malignant or pre-malignant tumor cell population(s). Thus, in some aspects, provided herein is a method for classifying lymphoproliferative disorder, e.g., a B-cell malignancy such as a B-cell lymphoma, a B-cell leukemia, or a plasma cell dyscrasia in a subject. Classifying the lymphoproliferative disorder may comprise inferring a cytogenetic classification of the lymphoproliferative disorder using single-cell RNA sequencing data.

The methods disclosed herein are capable of providing a classification (e.g., cytogenetic classification) of lymphoproliferative disorders, such as B-cell lymphomas, B-cell leukemias, and plasma cell dyscrasias (e.g., MM), with relatively few cells (e.g., less than 50 cells, less than 40 cells, less than 30 cells, less than 20 cells, or less than 10 cells). Thus, the methods disclosed here can be especially useful in the detection (and subsequent classification) of circulating tumor cells (CTCs) from a blood sample (or other liquid biopsy). The methods disclosed here can also be useful in the detection (and classification) of tumor cells from a bone marrow sample. The malignant or premalignant clonal cell population (e.g., a clonal population of CTCs) may comprise at least 5 or at least 10 cells to be classified by the present methods.

The present methods are especially advantageous in the detection and classification of pre-malignant conditions, such as MGUS, or in a post-treatment setting, where CTCs are more infrequent. A statistical model or machine learning algorithm as disclosed herein can be useful to probabilistically categorize an underlying lymphoproliferative disorder such as a B-cell malignancy based on a small data set, e.g., representative of less than 50 CTCs, or less than 20 CTCs. As shown herein, such a model or algorithm can provide a correct cytogenetic classification with as few as 5 or 10 CTCs.

### Training of the Statistical Model or Machine Learning Algorithm

A statistical model or machine learning algorithm can be trained using single-cell RNA sequencing data collected from subjects with cytogenetically classified lymphoproliferative disorders (e.g., MM) which are corresponding or related to the lymphoproliferative disorders the model is intended to classify (e.g., MGUS and SMM) and ground truth label data indicative of the cytogenetic classifications of the lymphoproliferative disorders of the subjects. The training of such models can also be done using data obtained by the methods disclosed herein.

In some aspects, a method of training a statistical model is provided to classify a lymphoproliferative disorder such as a B-cell malignancy (e.g., MM), the method comprising:
(a) cytogenetically classifying tumor tissue and/or CTC samples taken from subjects with the lymphoproliferative disorder and optionally related lymphoproliferative disorders (e.g., MGUS and SMM), to thereby generate ground truth label data that identifies cytogenetic classifications of the tumor tissue and/or CTC samples;
(b) carrying out single-cell RNA sequencing on cells from the tumor tissue and/or CTC samples, to thereby generate training input data; and
(c) training the model or machine learning algorithm, using the training input data and the ground truth label data, to classify the lymphoproliferative disorder (e.g., as MGUS, SMM, or MM).

The method may comprise training the model or machine learning algorithm, using the training input data and the ground truth label data, to identify expression signatures in single-cell RNA sequencing data that correspond to an underlying cytogenetic profile of the lymphoproliferative disorder identified by the ground truth label data, to thereby classify the lymphoproliferative disorder.

The term “related lymphoproliferative disorders” includes, e.g., precursor forms of the lymphoproliferative disorders (e.g., MGUS and SMM in the case of MM) that may progress in due course to the malignant form of the disorder.

Accordingly, a method disclosed herein may employ a machine learning algorithm that was trained on single-cell RNA sequencing data collected from cytogenetically classified tumor tissue and/or CTC samples obtained from patients suffering from a corresponding or related lymphoproliferative disorder, and ground truth label data indicative of the cytogenetic classification of the lymphoproliferative disorders of the patients.

The statistical model may comprise a multinomial logistic regression model and/or a support vector machine, or the like. Many other alternatives are available for the implementation of the methods described herein; however, a multinomial logistic regression model has been found to provide the best results.

The method may comprise training the statistical model using an iterative approach. Suitable iterative approaches include a leave-one-out method, in which some of the training data is retained (i.e., not used as an input) in an initial iteration. In subsequent iterations, the retained training data is used to as input to further calibrate the statistical model. For example, aggregating coefficients from the leave-one-out method may be used for calibration. Many other alternatives are available for the implementation of the methods described herein.

The collected sequencing data corresponding to malignant or pre-malignant tumor cell populations may be scored separately for each single cell for expression values of selected signature genes, as mentioned above. Expression values may be normalized, as explained above. Data (expression values) may be combined across groups of cells, such as by the calculation of median scores (or mode or mean scores).

Gene expression values for key oncogenes can be collected from the single-cell RNA sequencing data in a similar manner. Expression data may also be collected for one or more sets of genes (so called “expression signatures” or “gene signatures”) based on established knowledge about their association with cytogenetic abnormalities.

Expression values collected for key oncogenes and/or key expression signatures, such as mean and median expression values, can be used as features (training input data) to train a statistical model or machine learning algorithm, as discussed in further detail below.

The machine learning algorithm may be trained using expression values for one or more oncogenes that correlate with a cytogenetic profile related to the lymphoproliferative disorder (e.g., a B-cell malignancy such as MM) as features. The machine learning algorithm may be trained (or further trained) using expression values for one or more translocation partner genes that correlate with a cytogenetic profile related to the lymphoproliferative disorder as features. The machine learning algorithm may be trained (or further trained) using expression values for one or more gene expression signatures that correlate with a cytogenetic profile related to the lymphoproliferative disorder as features.

For example, for MM, key expression signatures may include expression data for genes including one or more of (or all of) CD-1, CD-2, MS, MF, and HY. Key oncogenes may include one or more of (or all of) CCND1, CCND2, CCND3, NSD2, FGFR3, MAF, MAFA, MAFB, and ITGB7. The latter are known to be associated and correlated with underlying IgH translocations in MM, and are accordingly also considered to be “translocation partner genes”. Such translocations and their effect on translocation partner genes are discussed, for example, in Zhan et al. Blood vol. (2006) 108 (6): 2020-2028, and Rajan et al. Blood cancer journal (2015) 5 (10): e365. Suitable gene expression signatures are provided in Table A.

For example, translocation t(11;14)(q13;q32) may be associated with expression of CCND1 (cyclin D1). Translocation t(4;14)(p16;q32) may be associated with expression of FGFR-3 and MMSET/NSD2/WHSC1. Translocation t(14;16)(q32;q23) may be associated with expression of C-MAF. Translocation t(14;20)(q32;q11) may be associated with expression of MAFB. Some IgH translocations may be associated with the expression of CCND3 (cyclin D3) in t(6;14).

A given gene, particularly a gene expressed at low levels such as a transcription factor that is upregulated as the result of a chromosomal translocation at an immunoglobulin locus, may not be detected in all cells that express that gene due to drop-outs that often occur with droplet-based single-cell RNA sequencing data. Mean gene expression at the tumor level can be used as a feature to overcome drop-out phenomena.

Translocation partner genes can be complemented by median signature levels of key cytogenetic signatures to further reduce the reliance on single genes. For translocations involving MAF transcription factors, such as t(14;16), t(14;20), and t(8;14), mean expression levels of ITGB7 can be included in the model, as there are cases in which little to no expression is detected for the underlying MAF transcription factor, yet ITGB7 is still highly expressed. In other words, the mean expression of ITGB7 can be used as a proxy to infer translocations of MAF transcription factors. For tumors with the t(4; 14) translocation, both NSD2 and FGFR3 levels can be included in the model, as well as median levels of the MS signature (see Table A), as FGFR3 expression is lost in a fraction of t(4;14) tumors. In some instances, the expression of both NSD2 and FGFR3 is used to infer the presence of a t(4;14) translocation.

Although exemplified herein for myelomas, namely MM and its precursor forms (MGUS and SMM), statistical models and machine learning algorithms as disclosed herein can also be provided for other cytogenetically well-characterized lymphoproliferative disorders, such as T cell lymphomas, and in particular B-cell malignancies such as B-cell lymphomas, B-cell leukemias, and plasma cell dyscrasias, using the methods disclosed herein.

The population of subjects used as sources for training data typically have a known underlying lymphoproliferative disorder such as a B-cell lymphoma, a B-cell leukemia, or a plasma cell dyscrasia, which may have been diagnosed by any suitable method. Typically, the subjects' lymphoproliferative disorders have been cytogenetically classified, such that the cytogenetic classification corresponding to the single-cell RNA sequencing data for each subject can be used as ground truth label data, optionally so that the impact of their data on the resultant model can be correctly selected/weighted. Such cytogenetic classification can be done in the first instance by established methods such as FISH and/or whole genome sequencing, and/or by the application of the present methods. It is advantageous for the source population of subjects to span a range of subcategories of a given lymphoproliferative disorder (e.g., representing each of the commonly observed cytogenetic events) and/or disease stages (e.g., early-stage lymphoma and late-stage lymphoma, and/or pre-treated or untreated lymphoma). In particular, several representative samples (e.g., at least 3, 5 or 10 samples, more typically at least 15, 30, or 50 samples) within each subcategory are typically required, especially if training by a leave-one-out method is used. A model may be trained with at least 10, at least 15, at least 20, at least 30, at least 40, at least 50, at least 75, at least 100, or at least 150 source subjects/patients.

Once a model has been appropriately trained, it can be used in the present methods to classify a lymphoproliferative disorder such as a B-cell lymphoma, a B-cell leukemia, or a plasma cell dyscrasia of a particular subject, based on appropriate input, such as single-cell RNA sequencing data corresponding to malignant or pre-malignant expanded clonal cell populations. Such input data used for classification by a statistical model or machine learning algorithm typically include scores for signature gene expression, oncogene expression, translocation partner gene expression, and combinations thereof.

Also provided is a computer program comprising computer program code configured to cause one or more physical computing devices to perform a method of training a statistical model to classify a lymphoproliferative disorder such as a B-cell lymphoma, a B-cell leukemia, or a plasma cell dyscrasia (e.g., MM) when the code is run. The method may comprise obtaining cytogenetic classifications of tumor tissue and/or CTC samples taken from subjects with the lymphoproliferative disorder (e.g., MM) and optionally related lymphoproliferative disorders (e.g., MGUS and SMM), to thereby generate ground truth label data that identifies cytogenetic classifications of the tumor tissue and/or CTC samples; obtaining single-cell RNA sequencing data for cells from the tumor tissue and/or CTC samples, to thereby generate training input data; and training the model or machine learning algorithm, using the training input data and the ground truth label data, to classify the lymphoproliferative disorder. The method may further comprise obtaining median expression scores of translocation partner genes from the tumor tissue and/or CTC samples, to thereby generate additional training input data, and training the model or machine learning algorithm, using the additional training input data and the ground truth label data, to classify the lymphoproliferative disorder.

Also provided is a computer readable storage medium (optionally non-transitory) having stored thereon a computer program as summarized above.

### Exemplary Method of Training a Statistical Model

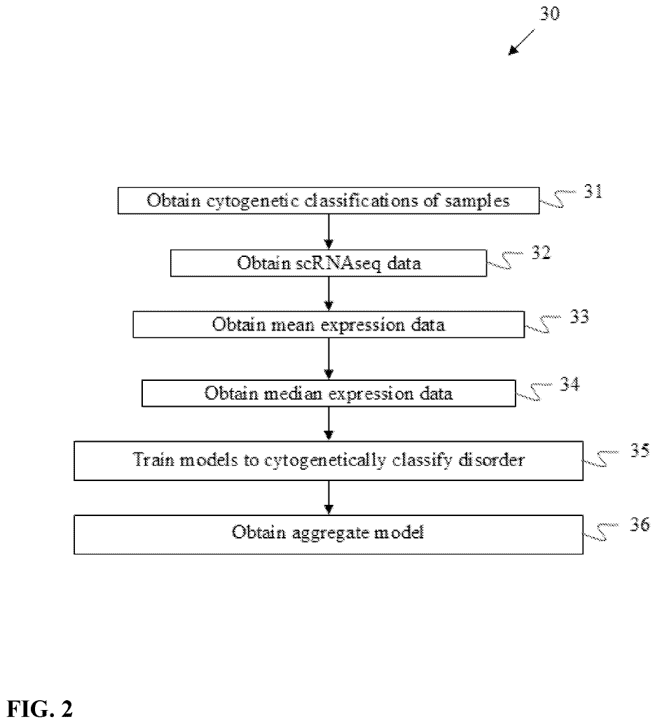

An exemplary method 30 of training a statistical model will now be described with reference to FIG. 2, for the case of multiple myeloma (MM).

Tumor tissue and/or circulating tumor cell (CTC) samples taken from subjects with MM are cytogenetically classified. The cytogenetic classifications are obtained 31 for use as ground truth data. In the present example, the classification is done by means of FISH; however, in other examples different techniques (such as whole genome sequencing) may instead be used. The cytogenetic classification of the samples indicates the cytogenetic profiles of the samples (that is, which chromosomal abnormalities such as translocations, focal or arm-level gains, amplifications, deletions etc. are exhibited by the samples). The cytogenetic classifications are used as ground truth data for the training of the statistical model. In some examples, the cytogenetic classification of the samples may have already occurred, and obtaining the cytogenetic classifications may comprise reading the classifications from memory or receiving the classifications from a network device.

Single-cell RNA sequencing is carried out on the samples to obtain 32 scRNAseq data for the samples. The scRNAseq data is used as training input data for the training of the statistical model. In some examples, the sequencing may have already been performed and obtaining 32 the scRNAseq data may comprise reading the data from a memory or receiving the data via a network device.

Mean expression data for key oncogenes (e.g., CCND1, CCND2, CCND3, NSD2, FGFR3, MAF, MAFA, MAFB, and ITGB7) is obtained 33. This data is used as training input data for the training of the statistical model. This data may be pre-calculated and may be obtained from a memory or received via a network device. In other examples, the mean expression data may be calculated based on the scRNAseq data.

Median expression data for key gene signatures (e.g., CD-1, CD-2, MS, MF, and HY etc.—see, e.g., Table A) is obtained 34. This data is used as training input data for the training of the statistical model. As with the mean expression data, the median expression data may be pre-calculated and may be obtained from a memory or received via a network device. Alternatively, the median expression data may be calculated based on the scRNAseq data.

A plurality of statistical models is trained 35 to cytogenetically classify MM based on scRNAseq data, mean expression data for key oncogenes, and median expression data for key signatures (see, e.g., Table A), using a leave-one-out approach.

An aggregate model is obtained 36 by aggregating coefficients from the plurality of leave-one-out models. In the present case, this is achieved by taking the mean of the coefficients from the plurality of leave-one-out models. In other examples, other known aggregation methods can be used, such as taking the median or modal coefficients from each leave-one-out model.

Steps 31-36 can be implemented as an independent computer-implemented method. Steps 33, 34 and 36 are optional—in some examples one or more of these steps may be omitted. For example, an aggregate model need not be obtained. Instead, the best (that is, most accurate) of the leave-one-out models may be selected for use.

Steps 31-34 can be performed in any order. In some examples, any combination of steps 31-34 may be performed in parallel.

In some instances, the models might not be trained using one or both of mean expression data for key oncogenes and median expression data for key signatures. In some instances, the model may be trained using only scRNAseq data as training input data.

The model may be trained to generate a probability for each of a plurality of classes and categorize the disorder into one or more of the classes if the probabilities for the one or more classes exceed a threshold. For example, the classes may be different translocations, and for each translocation a probability is generated based on the input data that indicates the likelihood that the translocation is present. The threshold probability for a single disorder sub-class can be set to be larger than 0.3, larger than 0.4, larger than 0.5, larger than 0.6, or larger than 0.7.

The model may further be trained using protein expression data as training input data.

The cytogenetic classification produced by the model may comprise the presence or absence of one or more of: driver mutation, loss of heterozygosity, single nucleotide variation (SNV), epigenetic abnormality, gene upregulation, gene downregulation, and alternative splicing.

Corresponding steps apply when training a machine learning algorithm.

### Use of the Statistical Model or Machine Learning Algorithm

A statistical model or machine learning algorithm may subsequently classify the lymphoproliferative disorder, e.g., the B-cell lymphoma, B-cell leukemia, or plasma cell dyscrasia (e.g., MM), by associating one or more detected malignant or pre-malignant tumor cell populations (represented by the data of the expanded clonal cell populations processed by a tumor cell profiling algorithm as described herein) with one or more cytogenetic classes. The associating may be based on the data of the expanded clonal cell populations (for example, the single-cell RNA sequencing data of the one or more malignant or pre-malignant tumor cell population(s), and optionally mean and median expression data). The data determined by the tumor cell profiling algorithm (for example, one or more of expression data for translocation partner genes and the presence of one or more CNVs) may be input to the statistical model.

For example, the tumor cell populations may be associated with one or more translocations. The one or more translocations may be selected from t(11;14), t(4;14), t(14;16), t(14;20), t(8; 14), and t(6;14)—e.g., in the case of MM. For each subject, there may be more than one identified distinct tumor cell population, and accordingly, more than one output classification for each, such as illustrated by the examples provided herein. Typically, the statistical model or machine learning algorithm can be adapted to output a classification based on the highest calculated probability, as long as said highest probability exceeds a certain threshold. For example, the minimum probability for a single disorder sub-class can be set to be larger than 0.3, larger than 0.4, larger than 0.5, larger than 0.6, or larger than 0.7. The probability value may be selected on the basis of the desired sensitivity. The lymphoproliferative disorder may be classified as belonging to the cytogenetic classification having the largest probability.

In some cases, classification may comprise a number of steps, and/or hierarchical assignment to cytogenetic classes, depending on the genetic implications of such classes. For example, in MM, the initial classification may comprise the presence or absence of a translocation. If a translocation is present, it may be assigned to a chromosomal translocation commonly associated with the lymphoproliferative disorder. If no translocation is determined (e.g., if the probability for a given translocation is determined to be less than 0.5 or other threshold value), the classification can be set as ‘no translocation’.

The collected data may be combined with other diagnostic data (e.g., FISH, karyotype, CNV inference, etc.) obtained for the same subject to improve categorization and/or provide additional useful information relating to the subject. These diagnostic data may be separate from, or integrated into, any other model or analysis of the present methods.

The statistical model or the machine learning algorithm may associate the one or more malignant or pre-malignant tumor cell population(s) with one or more copy number abnormalities if no translocation classification is made.

Accordingly, the statistical model or the machine learning algorithm may classify the lymphoproliferative disorder (e.g., MM) by associating the one or more malignant or pre-malignant tumor cell population(s) with one or more copy number abnormalities (e.g., hyperdiploidy) based on the single-cell RNA sequencing data of the one or more malignant or pre-malignant tumor cell population(s).

CNV inference can be used to determine other chromosomal abnormalities, especially where no translocation is detected. For example, in the case of MM, if CNV inference suggests amplification in at least two chromosomes, the MM can be classified as hyperdiploid.

Accordingly, the statistical model or the machine learning algorithm may associate the one or more malignant or pre-malignant tumor cell population(s) with one or more copy number abnormalities (e.g., hyperdiploidy) based on the inference of CNVs.

Secondary genomic abnormalities, such as specific deletions (e.g., Del13q, Del17p, Amp1q for MM) can also be determined with such approaches.

Accordingly, the statistical model may classify the lymphoproliferative disorder by associating the one or more malignant or pre-malignant tumor cell population(s) with one or more chromosomal abnormalities based on the single-cell RNA sequencing data of the one or more malignant or pre-malignant tumor cell population(s). The one or more chromosomal abnormalities may be selected from one or more of a 1q21 amplification, a 1p32 deletion, a 13q deletion, a 16q deletion, and a 17p deletion—e.g., in the case of MM.

Subclonal information can also be provided by such inference, which can be used as a measure of tumor heterogeneity. Subclonal analysis is particularly applicable when the sample analyzed is a blood sample comprising CTCs, as daughter subclones may be better represented in the CTC population than in a localized bone marrow or other biopsy, which may represent a biased geographical population. Advantageously, such analysis may provide information on the frequency of subclones and/or frequency of particular genomic abnormalities (such as high-risk abnormalities) in the subject.

Appropriate classification levels and subcategories can be selected for each lymphoproliferative disorder.

The method may further comprise detecting or inferring the presence of one or more of: driver mutation, loss of heterozygosity, single nucleotide variation (SNV), epigenetic abnormality, gene upregulation, gene downregulation, and alternative splicing.

The method may further comprise obtaining or receiving (as applicable) protein expression data from the sample obtained from the patients. The method may further comprise detecting or inferring the presence of one or more of: protein upregulation, protein downregulation, post-transcriptional, and one or more post-translational modifications.

The method may further comprise characterizing the disorder based on the classification, wherein characterization comprises one or more of: diagnosis, prognosis, determination of cancer subclassification, determination of tumor heterogeneity, monitoring disease progression, detection of subclones, identifying a treatment, monitoring treatment effect, and identification of disease risk.

As demonstrated in the examples provided below, the provided methods (including the computer-implemented methods) often detect lymphoid clones that have not been detected clinically (e.g., using FISH or other standard diagnostic methods). Thus, in some aspects, the provided methods may be used for screening subjects, such as subjects that belong to an at-risk population, including subjects above a certain age (e.g., older than 50 or older than 60 years of age).

### Mathematical Model

In a mathematical model (e.g., a statistical model or machine learning algorithm as described herein), a data matrix comprising single-cell RNA sequencing data is mapped onto labels corresponding to cytogenetic classifications of a lymphoproliferative disorder, e.g., a B-cell lymphoma, a B-cell leukemia, or plasma cell dyscrasia such as MM. Such classifications may relate to translocations characteristic of subcategories of a lymphoproliferative disorder, including subcategories of B-cell lymphomas, B-cell leukemias, and plasma cell dyscrasias. In other models, a data matrix comprising single-cell RNA sequencing data is mapped onto labels corresponding to classifications of clonal cell populations, which classifications relate to one or more of clonal cell populations, expanded clonal cell populations, premalignant and/or malignant clonal cell populations. Suitable statistical models or machine learning algorithms may include, e.g., neural networks or linear classifiers.

Models such as those described herein can be generated using scores for expression of selected genes as input to train a machine learning model, such as a multivariate logistic regression model. A model can be trained and verified iteratively, such as following a leave-one-out approach. An aggregate model can be prepared by aggregating coefficients from the leave-one-out models, such as by taking the mean, median or modal coefficients from each leave-one-out model.

The probability for each class may be computed using the equation:

$${p}_{j} = \frac{exp ( {b}_{0 , j} + {b}_{1 , j} * {x}_{1} + ⋯ + {b}_{i , j} * {x}_{i} )}{1 + exp ( {b}_{0 , j} + {b}_{1 , j} * {x}_{1} + ⋯ + {b}_{i , j} * {x}_{i} )} ,$$

where j corresponds to a particular class, i corresponds to a particular feature, and b corresponds to a coefficient specific for feature i and class j.

### Tumor Burden

The methods described herein can be used to determine tumor burden in a tissue (e.g., bone marrow) and blood (i.e., circulating tumor cell (CTC) burden) sample of a subject diagnosed with a lymphoproliferative disorder such as a B-cell malignancy (e.g., a B-cell lymphoma, a B-cell leukemia, or a plasma cell dyscrasia such as MM). Information on tumor burden can be used for risk stratification and therefore can inform management of the lymphoproliferative disorder in the subject.

Tumor burden can be determined by dividing the number of malignant cells expressing the one or more lineage marker (e.g., malignant plasma cells) by the total number of cells expressing the one or more lineage markers (e.g., the number of malignant and non-malignant plasma cells). Alternatively, tumor burden can be determined by dividing the number of malignant cells expressing the one or more lineage marker (e.g., malignant plasma cells) by the total number of cells in the population for which single-cell RNA sequencing data was obtained (e.g., the cells enriched for a cell type comprising tumoral cells using a cell surface marker).

Accordingly, also provided is a computer-implemented method for determining tumor burden in a subject suffering from a lymphoproliferative disorder such as a B-cell malignancy (e.g., a B-cell lymphoma, a B-cell leukemia, or a plasma cell dyscrasia such as MM), wherein the method comprises:
(a) obtaining single-cell RNA sequencing data (including, e.g., single-cell B cell receptor (BCR) V(D)J sequencing data) for a population of cells, wherein the population of cells was obtained from a sample obtained from the subject by enriching the sample for a cell type comprising tumoral cells using a cell surface marker;
(b) applying a tumor cell profiling algorithm to the single-cell RNA sequencing data; wherein the algorithm:
   i. identifies cells expressing one or more lineage markers of the cell type comprising the tumoral cells,
   ii. identifies among the cells expressing the one or more lineage markers one or more expanded clonal cell populations (e.g., based on the BCR V(D)J sequencing data);
   iii. determines the number of cells in each of the one or more expanded clonal cell populations;
   iv. categorizes each of the one or more expanded clonal cell populations as malignant or pre-malignant if expression of a pre-determined set of oncogenes and tumor surface markers specific to the lymphoproliferative disorder (e.g., the B-cell malignancy) is detected; and
   v. determines the tumor burden by dividing the number of malignant or pre-malignant cells by (1) the total number of cells expressing the one or more lineage markers, or (2) the total number of cells in the population for which single-cell RNA sequencing data was obtained.

### Computational Implementation

The described methods may, in part, be implemented on a computer. The computer may receive first data relating to single-cell RNA sequencing data obtained from a sample collected from a subject. Typically, the single-cell RNA sequencing data are enriched for data sets from mononuclear cells and/or subsets therefore (e.g., plasma cells, B cell, or T cells), e.g., by performing the enrichments, selection or isolation steps disclosed elsewhere herein prior to the step of single-cell RNA sequencing.

The computer may also receive meta-data, such as the subject's diagnosed or suspected lymphoproliferative disorder, and/or diagnostic information such as previously determined immunoglobulin isotype or cytogenetic profile. The cytogenetic profile may comprise information on one or more translocations and/or one or more copy number abnormalities (e.g., hyperdiploidy).

The computer may obtain a statistical model, analyze the single-cell RNA sequencing data with the statistical model and provide, as an output of the statistical model, a score. The statistical model may take into account the meta-data. The statistical model may comprise one or models selected from: a linear classification model, linear discriminant analysis, logistic regression, multinomial logistic regression, multivariate adaptive regression splines, naive Bayes, neural network, support vector machine, functional tree, LAD tree, Bayesian network, elastic net regression, and random forest.

The score may be suitable for at least one of: identifying the subject as having a lymphoproliferative disorder (e.g. a B-cell malignancy such as a B-cell lymphoma, a B-cell leukemia, or a plasma cell dyscrasia); identifying the subject as having a particular subcategory of a lymphoproliferative disorder; identifying the subject as having a lymphoproliferative disorder corresponding to a particular cytogenetic classification; and/or identifying the subject as being suited to a particular therapy.

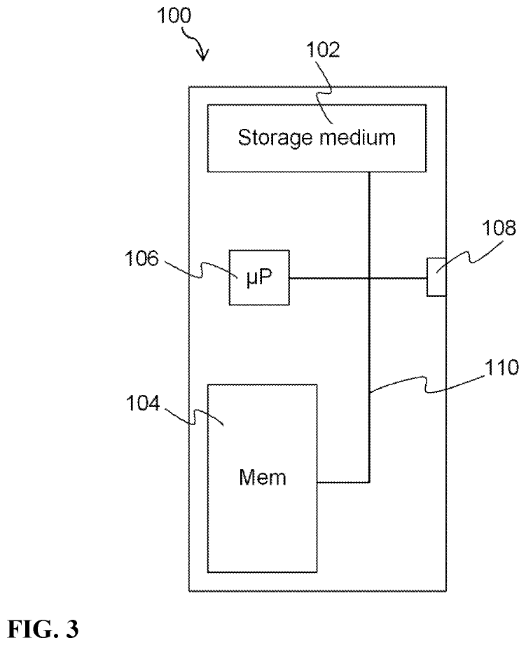

FIG. 3 of the accompanying drawings schematically illustrates an exemplary computer system 100 upon which a computer program as disclosed herein may run. The exemplary computer system 100 comprises a computer-readable storage medium 102, a memory 104, a processor 106 and one or more interfaces 108, which are all linked together over one or more communication busses 110. The exemplary computer system 100 may take the form of a conventional computer system, such as, for example, a desktop computer, a personal computer, a laptop, a tablet, a smart phone, a server, a mainframe computer, and so on.

The computer-readable storage medium 102 and/or the memory 104 may store one or more computer programs (or software or code) and/or data (including but not limited to: the first data, ground truth label data, training input data, and the statistical model). The computer programs stored in the computer-readable storage medium 102 and/or the memory 104 may include computer programs that, when executed by the processor 106, cause the processor 106 to carry out a method as disclosed herein. The computer-readable storage medium 102 and/or the memory 104 may be a non-transitory computer readable storage medium. The computer-readable storage medium 102 and/or the memory 104 may be one or more memory chips or memory blocks implemented within the processor, magnetic media such as hard disk or floppy disks, or optical media such as for example DVD and the data variants thereof, CD.

The processor 106 may be any data processing unit suitable for executing one or more computer readable program instructions, such as those belonging to computer programs stored in the computer-readable storage medium 102 and/or the memory 104. The processor 106 may include one or more of: general purpose computers, special purpose computers, microprocessors, digital signal processors (DSPs), application specific integrated circuits (ASIC), gate level circuits and processors based on multi-core processor architecture, as non-limiting examples. As part of the execution of one or more computer-readable program instructions, the processor 106 may store data to and/or read data from the computer-readable storage medium 102 and/or the memory 104. The processor 106 may comprise a single data processing unit or multiple data processing units operating in parallel or in cooperation with each other. The processor 106 may, as part of the execution of one or more computer readable program instructions, store data to and/or read data from the computer-readable storage medium 102 and/or the memory 104.

The one or more interfaces 108 may comprise a network interface enabling the computer system 100 to communicate with other computer systems across a network. In some examples, the computer system 100 may obtain the first data, ground truth label data, training input data, and/or the statistical model via the network. The network may be any kind of network suitable for transmitting or communicating data from one computer system to another. For example, the network could comprise one or more of a local area network, a wide area network, a metropolitan area network, the internet, a wireless communications network, and so on. The computer system 100 may communicate with other computer systems over the network via any suitable communication mechanism/protocol. The processor 106 may communicate with the network interface via the one or more communication busses 110 to cause the network interface to send data and/or commands to another computer system over the network. Similarly, the one or more communication busses 110 enable the processor 106 to operate on data and/or commands received by the computer system 100 via the network interface from other computer systems over the network.

The interface 108 may alternatively or additionally comprise a user input interface and/or a user output interface. The user input interface may be arranged to receive input from a user, or operator, of the system 100. The user may provide this input via one or more user input devices (not shown), such as a mouse (or other pointing device, track-ball or keyboard. The user output interface may be arranged to provide a graphical/visual output to a user or operator of the system 100 on a display (or monitor or screen)(not shown). The processor 106 may instruct the user output interface to form an image/video signal which causes the display to show a desired graphical output. The display may be touch-sensitive enabling the user to provide an input by touching or pressing the display.

The interface 108 may alternatively or additionally comprise an interface to a measurement system, for measuring single-cell RNA sequencing data for a sample obtained from the subject (for example, a tumor tissue or CTC sample, or a sample of cells enriched by selecting for mononuclear cells and/or a subset thereof).

In general, the various examples may be implemented in hardware or special purpose circuits, software, logic or any combination thereof. For example, some aspects may be implemented in hardware, while other aspects may be implemented in firmware or software which may be executed by a controller, microprocessor or other computing device, although these are not limiting examples. While various aspects may be illustrated as block diagrams, it is well understood that these blocks—may be implemented in, as non-limiting examples, hardware, software, firmware, special purpose circuits or logic, general purpose hardware or controller or other computing devices, or some combination thereof.

A single processor or other unit may fulfil the functions of several items recited in the claims. The functions may be performed in a single integrated electronic device, or the functions may be distributed across different discrete devices. For example, some functions may be performed by a remote service accessed via a wired or wireless network connection. A computer program may be stored/distributed on a suitable medium, such as an optical storage medium or a solid-state medium supplied together with or as part of other hardware, but may also be distributed in other forms, such as via the Internet or other wired or wireless telecommunication systems.

Also provided is a computer program comprising computer program code configured to cause one or more physical computing devices to perform any of the computer-implemented methods summarized above when the code is run. Also provided is a computer readable storage medium (optionally non-transitory) having stored thereon a computer program as summarized above.

### EXAMPLES

It will be readily apparent to those skilled in the art that other suitable modifications and adaptations of the provided methods and computer programs described herein may be made using suitable equivalents. The following examples are included for purposes of illustration only and are not intended to be limiting.

### Example 1. Single-Cell RNA Sequencing of Paired Bone Marrow and Peripheral Blood Cells

Single-cell RNA sequencing, including targeted amplification of V(D)J transcripts to perform B cell receptor (BCR) sequencing, was performed on paired bone marrow and peripheral blood (PB)-derived CD138+ plasma cells from 73 patients (MGUS: n=11; SMM: n=45; Newly Diagnosed Multiple Myeloma (NDMM): n=17) and 28 healthy donors.

Where relevant, the SMM group of patients were further stratified based on the “20-2-20” prediction model developed by the International Myeloma Working Group (IMWG), which stratifies patients based on BM plasma cell infiltration (>20%), the amount of M protein in the serum (>2 g/dL), and the free light-chain ratio (>20); this rendered a cohort of 21 patients with low-risk SMM (LRSMM), 9 with intermediate-risk (IRSMM) and 15 with high-risk SMM (HRSMM). Patients in the study had routine clinical cytogenetics and FISH analysis performed on their bone marrow biopsies, and serum protein electrophoresis (SPEP), serum free light chain (SFLC) assay and additional commonly performed blood laboratory tests performed on their peripheral blood samples in clinical-grade pathology labs.

Samples from 28 healthy donors (13 paired BM and PB, 15 BM only) were also collected. Participants were considered healthy for the purposes of this study based on a negative screening result for the presence of abnormal monoclonal protein by MALDI-TOF mass spectrometry, and no past or known current diagnosis of cancer.

### Sample Processing, Cell Enrichment and Single-Cell RNA Sequencing

Whole bone marrow aspirates (5-20 mLs) and peripheral blood (30 mLs) were drawn into EDTA preservative tubes and kept at 4° C. before being processed within a 6-hour period of time from collection. Peripheral blood mononuclear cells were gently isolated from whole blood samples using a two-step protocol; 1) erythrocytes were aggregated and sedimented and 2) non-PBMCs were magnetically labeled with a cocktail of biotin-conjugated monoclonal antibodies targeting granulocytes and residual erythrocytes (Miltenyi Biotec, Cat #130-115-169). The magnetically labelled non-PBMCs were then separated from the sample by retaining them within a MACS column of an autoMACS Pro separator, while unlabeled target PBMCs remained untouched for subsequent plasma cell enrichment.

Bone marrow mononuclear cells were isolated from aspirates using density gradient centrifugation. Briefly, the bone marrow was filtered to remove any clots or bone debris, diluted 1:3 with 1× Phosphate-Buffered Saline (PBS), and gently poured above 15 mLs of Ficoll-Paque density gradient medium within a SepMate™ PBMC isolation tube (StemCell Technologies, Cat #85450). After centrifugation at 1200 rcf/g for 15 minutes, PBMCs were poured away from unwanted cells and washed twice with ice cold 1×PBS prior to plasma cell enrichment.

Plasma cells were enriched from both the bone marrow and the peripheral blood samples using CD138 magnetic bead separation, where BM samples underwent single-column positive selection and PB samples underwent double-column positive selection due to the lower frequency of CTCs and desire for higher purity capture of CTCs within the PB (Miltenyi Biotec, Cat #130-097-614). The resulting cells were then loaded onto the Chromium Controller (10× Genomics) to perform single cell partitioning and barcoding.

Libraries were prepared with the Chromium Single Cell 5′ Gene Expression and V(D)J enrichment kit by 10× Genomics and sequenced on NovaSeq S4 flow cells (Illumina).

### Data Filtering

CellRanger mkfastq (v5.0.1) was used to generate FASTQ files. Gene expression matrices were generated by CellRanger count (v6.0.1) with the hg38 genome reference provided by 10× Genomics. To remove ambient RNA, CellBender (v0.2.0) was run on the gene expression matrices with the target false positive rate cutoff of 0.01. Poor quality cells with >15% mitochondrial gene expression, either <200 detected genes, >5,000 detected genes, <400 UMIs, or >50,000 UMIs were filtered out. Three doublet tools, Scrublet (v0.2.3), scDblFinder (v1.8.0), and scds (1.10.0) were used to calculate multiplet scores and heterotypic multiplets that clustered separately and showed expression of markers specific to several cell types were excluded from downstream analysis. CellRanger vdj (v6.0.1) was used with the flag of “-chain=IG” on FASTQ files with the hg38 VDJ reference file provided by 10× Genomics.

### Plasma Cell Identification and Clonotyping

Plasma cells were first identified on the basis of expressing key lineage markers, such as SDC1 (encoding CD138), CD38, XBP1, PRDM1, IRF4, and TNFRSF17 (encoding BCMA). Cells with expression barcodes that were determined to correspond to plasma cells were considered for downstream analysis.

All V(D)J contigs detected by CellRanger (v6.0.1) were then processed to identify a single heavy and light chain per cell barcode, based on Unique Molecular Identifier (UMI) support. For each patient, clonotypic data from multiple samples or aliquots were combined and harmonized clonotypes were identified anew based on the frequency of unique CDR3 amino-acid (AA) sequences. Occasionally, clonotypes with only a heavy or light chain were identified; such single-chain clonotypes were collapsed with the matching two-chain clonotype that had the highest frequency. Clones were sorted based on their cell count and expanded clones were identified as clones with at least 20 cells which are at least twice the size of the next clone in order of frequency.

### Example 2: Identification and Comparison of Normal and Malignant Clones

### Clone Identification

Expanded clones were manually reviewed to determine whether they were normal or malignant. An expanded clone was considered malignant if one or more of the following criteria was met: (i) its immunoglobulin isotype matched that reported clinically through serum protein immunofixation (IFX), (ii) key multiple myeloma oncogenes (for example, CCND1, MAF, WHSC1, etc.) were expressed and in accordance with the tumor's cytogenetics as determined by FISH, and (iii) key immunophenotypic markers of plasma cell malignancy (NCAM1, CD19, PTPRC, etc.) were expressed in the population. In some instances, no information concerning the tumor's cytogenetics was available (e.g., because FISH failed to detect a cytogenetic hallmark such as an IgH translocation). In some instances, a tumor was clinically undetected and therefore no information concerning the tumor's cytogenetics or immunoglobulin isotype was available.

Secondary expanded clones (typically with <50-100 cells) that clustered with normal plasma cells (typically in the peripheral blood compartment), had not been clinically detected as monoclonal protein spikes in serum protein electrophoresis (SPEP) and IFX, and did not show high levels of expression of key MM oncogenes or immunophenotypic markers were considered normal. In three patients, a clinically undetected second tumor was identified; in the absence of IFX or FISH data to compare against, these clones were considered malignant based on clustering separately from normal plasma cells and the expression of key MM oncogenes (namely, CCND1 and MAF) and immunophenotypic markers of malignancy. Specifically, the clones' expression data were sufficiently different from normal cells such that when it was attempted to represent all cells in a 2-D embedding (for example, UMAP), they formed a distinct cluster far away from normal plasma cells.

### Determining Copy Number Variants

Copy number variants (CNVs) were inferred using Numbat (v1.1.0) (Gao T et al, cited supra). Specifically, allelic data was collected from plasma cells (including malignant and normal ones) and B cells from both the BM and the PB to ensure that the frequency of CNVs and particularly, clonal deletions, would not negatively impact genotyping and phasing in samples with high tumor purity.

A custom panel of 1.2K healthy donor plasma cells (based on the single-cell RNA sequencing data obtained from the 28 healthy individuals, see Example 1) was used as expression reference, to correct for cell type-specific expression patterns, such as immunoglobulin expression, which otherwise result in artifactual CNV inference on chromosomes 2, 14, and 22. Ultimately, CNVs were inferred on a combined list of tumor cell barcodes from the BM and the PB for each patient to increase sensitivity. Four patients with 30-40K tumor cells across samples and compartments had to be downsampled prior to running Numbat; specifically, if a sample had <3K tumor cells, all tumor cells were kept, otherwise 3K tumor cells were randomly sampled for this run.

### Clone Detection and Phylogenetic Analyses

For clone detection and phylogenetic analyses, cells with <7.5K unique molecular identifiers (UMIs)/cell, <500 genes/cell, and >5% mitochondrial transcripts were removed from consideration. For each cell in the sample being analyzed, an expression-based probability that a given CNV exists in that cell was determined using the single-cell RNA sequencing data, and an allele-based probability that the CNV exists in that cell was determined using the single-cell RNA sequencing data. This process was performed for each CNV in question. For each cell, a joint CNV probability was determined for each CNV using the expression-based probability for that CNV in that cell and the allele-based probability for that CNV in that cell. The joint CNV probabilities calculated for all CNVs and cells were used to compute a Euclidean distance matrix, followed by agglomerative hierarchical clustering with complete linkage to identify subclones in the sample. The number of subclones was determined empirically based on the dendrogram, and subclones making up<20% of the tumor were removed from consideration, as they typically represented noise. In some examples, the subclones can be identified as the two top level cluster in the dendrogram. In some examples, subclones can be identified by lower-level clusters in the dendrogram. In the present example agglomerative hierarchical clustering was used; however, in different examples other techniques may be used, such as Bayesian clustering.

In cases where only one subclone was >20% of the sample, the top two subclones in frequency were considered. Subclone-level probabilities were then computed based on the joint CNV probability for each CNV in the respective subclone. In particular, the subclone level probability for a given CNV in a given subclone was determined to be equal to the median joint CNV probability of that subclone (in other words, the median of the joint CNV probabilities of the cells in that subclone for the given CNV). CNVs were called (that is, identified as being present in that subclone and thus part of the subclone's CNV profile) if the median joint CNV probability of that CNV being present was >0.65. CNVs not called in any potential subclone (that is, not identified as being present in any of the subclones) were removed from consideration. The threshold median joint CNV probability at which a CNV is called was set according to the desired balance between sensitivity and specificity. In the present example the threshold was set to 0.65. In other examples, the threshold may be 0.3, or 0.4, or 0.5, 0.6 or 0.8. In some examples the threshold might not be more than 0.8. The threshold may be in range 0.3-0.8, or in the range 0.5-0.8. The distributions of joint CNV probabilities for all CNVs in all subclones were manually reviewed and, in two cases, two CNVs were force-called in one of the subclones, as the joint CNV probability distribution of that subclone significantly favored their presence in the subclone despite a median joint CNV probability <0.65. Clone-level CNV profiles were then used for phylogenetic tree inference via maximum parsimony analysis, as implemented in the R package phanghorn.

### Cell Comparison—Peripheral Blood Samples

After filtering for cell quality, a total of 1,251,825 single CD138+ plasma cells were obtained for subsequent analyses, comprising 719,956 malignant and 323,670 normal plasma cells from the BM and 84,912 CTCs (i.e., malignant cells identified from PB samples) and 123,129 normal plasma cells from the PB.

Using UMAP to visualize the single-cell expression data, normal plasma cells grouped together, while malignant cells from patients clustered separately in a patient-specific manner, reflecting the unique transcriptional profile of each tumor. Malignant and normal plasma cells separated by compartments, with CTCs clustering closest to their matched BM tumor cells, indicating transcriptional similarity but not complete uniformity. CTCs shared the dominant BCR clonotype of the BM-resident tumor cells in all cases.

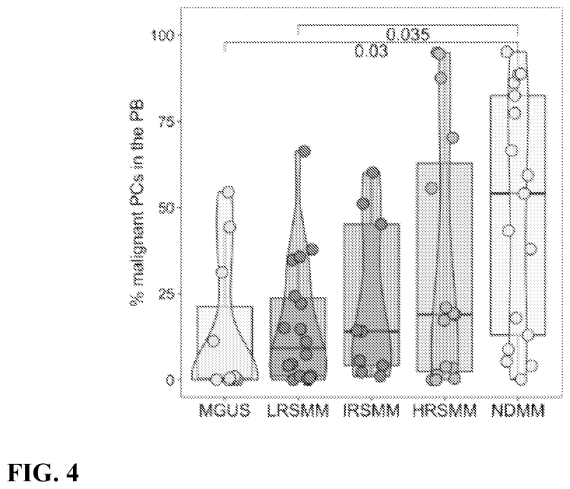

CTCs were detected in 90% (n=66/73; 95% CI: 81-96%) of patients in the cohort and 88% (n=59/66; 95% CI: 79-95%) of those had at least 10 CTCs detected. The patients who had no CTCs detected were all patients with MGUS or SMMs, who are known to have fewer tumor cells circulating in peripheral blood. The number of CTCs analyzed per sample ranged from 1 to 27,388, with a median of 204 CTCs per sample. The median proportion of malignant plasma cells out of all plasma cells in the PB increased significantly with disease progression from MGUS (0.62%) to low-risk SMM (LRSMM, 7.88%), intermediate-risk SMM (IRSMM, 16.21%), high-risk SMM (HRSMM, 20.02%), and NDMM (66.6%) (FIG. 4).

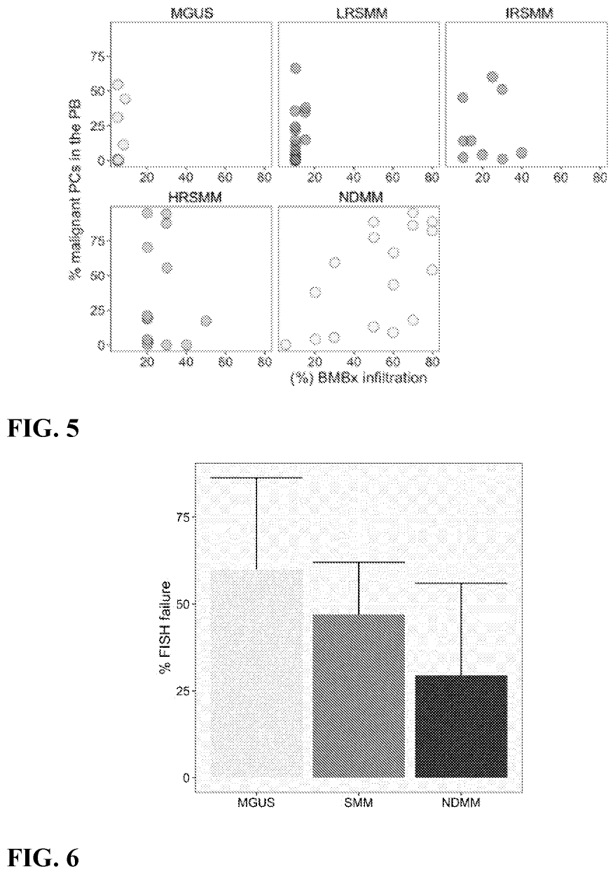

These results suggest sequencing-based CTC enumeration captures prognostically relevant differences in tumor burden, with the added benefit of also providing comprehensive transcriptomic profiling in tandem. There was a modest positive correlation between BM infiltration and CTC burden (Pearson's, r=0.53, p=2.2e-06), however, CTC levels were variable even within groups of patients with similar tumor burden and clinical staging. FIG. 5 illustrates this, showing a scatterplot demonstrating the correlation between % plasma cells present within BM aspirates obtained from clinical pathology reports (x-axis) compared to percent CTCs present in the PB (y-axis), stratified by disease stage and risk status (top label) (r=0.67). This suggests that other factors beyond just BM burden may influence CTC burden.

### Example 3: Multinomial Logistic Regression Model

In the analyzed cohort, 60% (95% CI: 27.4-86.3) of patients with MGUS, 47% (95% CI: 32-62) of patients with SMM, and 29% (95% CI: 11-56) of patients with NDMM were unclassified by FISH (FIG. 6). To expand the clinical utility of single-cell RNA sequencing (scRNA-seq) of CTCs past enumeration, we explored the feasibility of using small numbers of CTCs instead of BM tumor cells for the detection of cytogenetic events.

This example demonstrates that a multinomial logistic regression model trained on single-cell RNA sequencing data obtained from previously classified bone marrow (BM) samples is highly accurate in classifying matched single-cell RNA sequencing data of circulating tumor cells (CTC).

Malignant cells from each patient's compartment(BM or PB) were scored separately for key MM signatures (CD-1, CD-2, MS, MF, HY) reported by Zhan et al. (supra—see Table A) using Scanpy's score_genes function. After scoring, the median signature scores were calculated across cells from each compartment in each patient. Median signature scores as well as mean log1p-transformed gene expression values for key MM oncogenes (CCND1, CCND2, CCND3, NSD2, FGFR3, MAF, MAFA, MAFB) per patient and compartment were used as features in a multivariate logistic regression model to detect IgH translocations. These features were selected based on established knowledge about their association with underlying IgH translocations in MM. These translocation partner genes were demonstrated as upregulated in accordance with translocation type identified by FISH in both BM tumor cells and CTCs.

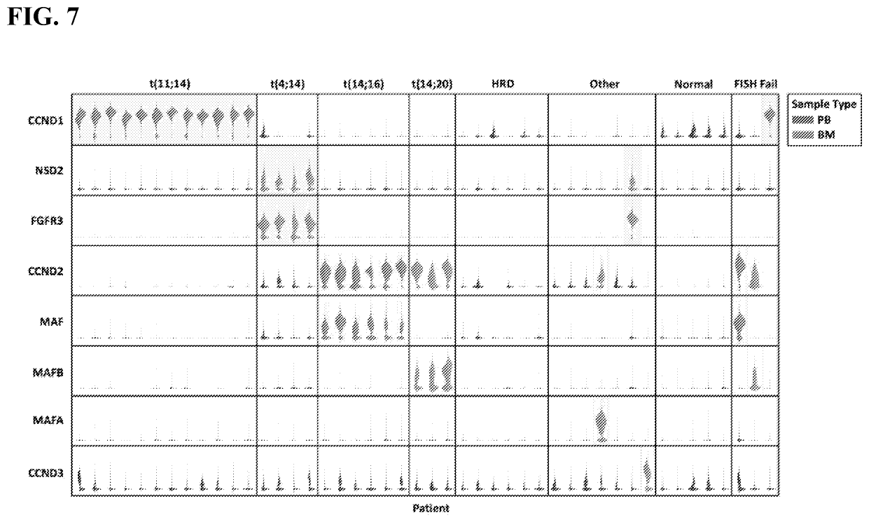

FIG. 7 illustrates this, showing a Split violin plot with expression levels of common IgH translocation partner genes (left label) present in BM tumor cells (right-side of violin) and CTCs (left-side of violin). Each column or violin plot represents an individual patient from the cohort that had at least 50 tumor cells in the BM and 50 CTCs captured in the paired PB for clear visualization. All patients were ordered based on their fluorescence in situ hybridization (FISH) clinical pathology results obtained from the BM (top label), which served as the ground truth data for existence of MM chromosomal translocations. Normal or inconclusive FISH results due to an insufficient number of tumor cells available for testing used whole genome sequencing (WGS) of BM tumor cells when available to identify translocation events. Patterns of expression characteristic of each underlying translocation can be seen, as can similarities in expression between BM tumor cells and CTCs for each patient.

A multinomial logistic regression model was trained on single-cell RNA sequencing data obtained from 53 BM samples of patients who were cytogenetically classified with either an IgH translocation or hyperdiploidy (HRD) either by FISH (n=41) or by research-level whole-genome sequencing (n=33), when that was available. The data were obtained as described in Examples 1 and 2.

Established RNA signatures that correspond to underlying cytogenetic profiles (CD-1, CD-2, HP, MS, and MF) were used as features (see Table A). In addition, key translocation partner genes (CCND1, CCND2, CCND3, NSD2, FGFR3, MAF, MAFA, MAFB) were used as features. Hyperdiploidy, as determined by FISH or WGS, was used as a reference to ensure no translocation was present in a sample.

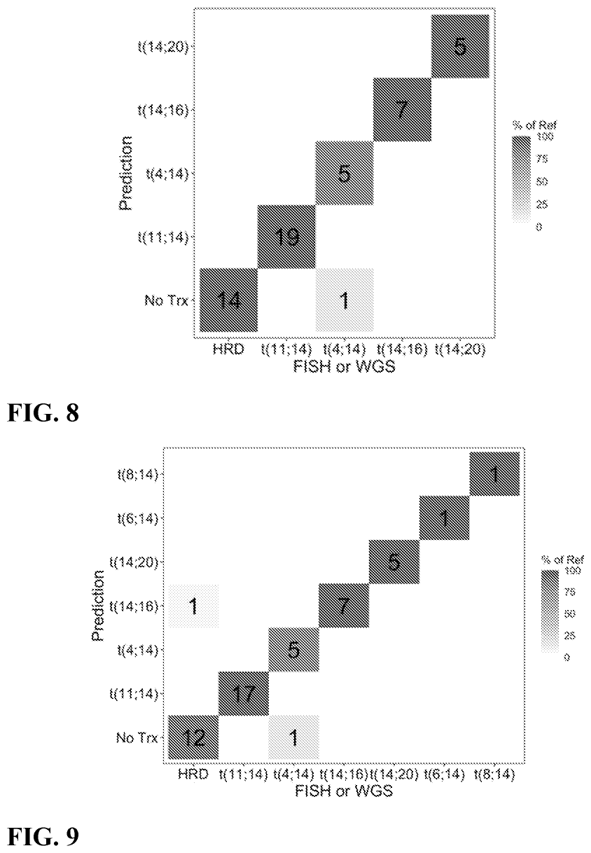

To limit overfitting, leave-one-out cross-validation was performed on the aggregated results from the 53 held-out samples. Excluding two cases with t(6;14) and t(8;14), remarkable performance was observed. These two cases were the only cases with such translocation for which performance could not be assessed as the models tested on them would by definition not have been trained on similar cases. 50 out of 51 cases were classified correctly (FIG. 8, a confusion matrix visualizing the performance of 51 leave-one-out models on the 51 hold-out cases. Within each tile, the absolute number of cases is visualized). The one case with t(4;14) that was misclassified as “No Trx” (i.e., no translocation) was an edge case with no FGFR3 expression and the lowest NSD2 expression out of all cases with t(4;14).

Coefficients from the 53 leave-one-out models were aggregated by taking the median and used to generated an aggregate model, whereby the probability for each class is computed using the equation:

$${p}_{j} = \frac{exp ( {b}_{0 , j} + {b}_{1 , j} * {x}_{1} + ⋯ + {b}_{i , j} * {x}_{i} )}{1 + exp ( {b}_{0 , j} + {b}_{1 , j} * {x}_{1} + ⋯ + {b}_{i , j} * {x}_{i} )} ,$$

where j corresponds to a particular class and i corresponds to a particular feature. Subsequently, the tumor is classified as the class with the maximum probability, provided that probability is larger than 0.5. Alternatively, it is classified as “No Trx” (no translocation).

Applying this bone marrow-trained aggregate model on single-cell RNA sequencing data of matched CTC samples, strikingly high accuracy was observed (n=49/50 or 98%; 95% CI: 88-100). FIG. 9 shows a confusion matrix visualizing the performance of the aggregated bone marrow-trained model on CTC samples. Within each tile, the absolute number of cases was visualized. The sole misclassified case was, again, the edge t(4;14) case with no FGFR3 expression and minimal upregulation of NSD2. The accuracy of classification was assessed based on the performance of the leave-one-out models on the corresponding held-out BM samples. The aggregate model's performance was assessed based on the accuracy of classification for CTC samples from patients with FISH/WGS-detected IgH translocation.

In one patient, two different myelomas were discovered. Both were correctly classified. One was classified as “No Trx” and was identified as hyperdiploid by FISH and WGS. Another one was classified as t(14;16), but was too small to be detected by FISH or WGS. Overall, the CTC classifier was able to classify the majority of cases that had previously unknown cytogenetic backgrounds (i.e., were unclassified by FISH).

Cases classified as “No Trx” by the multinomial logistic regression model were further assessed. Such cases were classified either as hyperdiploid (HRD) based on whether CNV inference using Numbat revealed amplification in at least two chromosomes, according to the official definition of hyperdiploidy in MM (>=48 chromosomes). Cases that did not have a translocation or HRD were classified as “Other”.

### Example 4: Single-Cell RNA Sequencing is Superior to FISH

This example illustrates that single-cell RNA sequencing can have greater sensitivity than FISH in correctly classifying patients suffering from plasma cell dyscrasias. It is also superior to FISH in identifying high-risk genetic abnormalities in these patients.

The reliability of capturing copy number abnormalities using scRNA-seq was examined, and it was demonstrated that it recapitulates results obtained from BM FISH clinical testing.

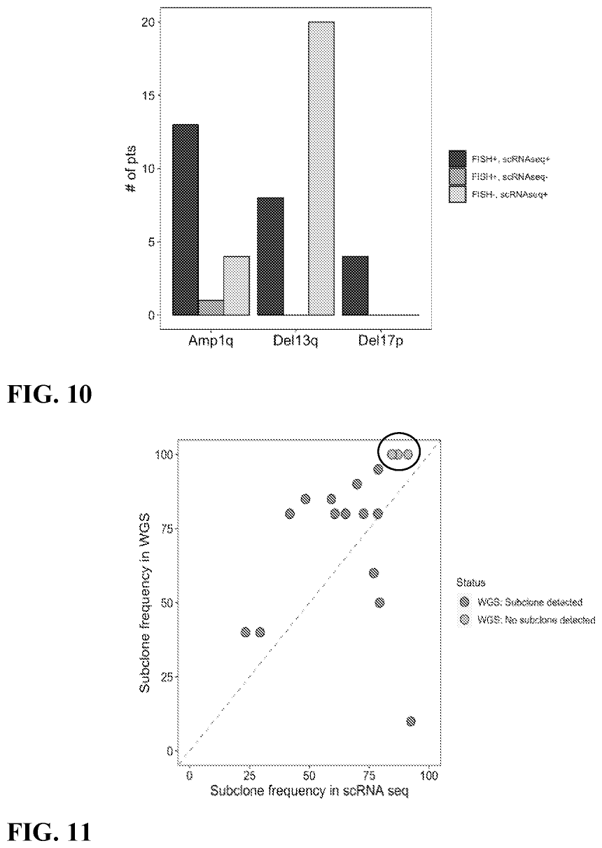

In terms of secondary genomic abnormalities, all 4 patients identified as having Del17p by FISH were shown to have Del17p (sensitivity of 100%; 95% CI: 40-100). Moreover, all 8 patients identified as having Del13q by FISH were shown to have Del13q (sensitivity of 100%; 95% CI: 60-100). Surprisingly, 20 additional patients were shown to have previously undetected Del13q, suggesting the sensitivity of FISH compared to single-cell RNA seq is less than 30% for this abnormality (FIG. 10).

Out of 14 patients identified as having Amp1q by FISH, 13 were shown to have Amp1q (sensitivity of 93%; 95% CI: 64-100). Notably, single-cell RNA sequencing detected Amp1q in 4 more patients, suggesting that the sensitivity of FISH is 77% (95% CI: 50-92) for this particular abnormality.

This example demonstrates that a multinomial logistic regression model using single-cell RNA sequencing data can reliably detect both IgH translocations/HRD as well as secondary high-risk genomic abnormalities in significantly more patients compared to FISH.

### Example 5: Single-Cell RNA Sequencing Outperforms Whole Genome Sequencing

This example illustrates that using a multinomial logistic regression model for the analysis of single-cell RNA sequencing results in a more accurate estimate of subclone sizes in patients suffering from multiple myeloma than whole genome sequencing (WGS).

WGS is capable of detecting mutations in addition to CNVs and translocations. Furthermore, it may show superior sensitivity when paired with prior tumor/plasma cell enrichment. Therefore, efforts have been made to replace FISH with bulk DNA sequencing using either targeted gene panels or whole-genome sequencing. Bulk DNA sequencing approaches estimate copy number based on the number of reads mapping to a specific region compared to the number of reads in a matched normal sample. Such approaches can work well, but may not be perfectly accurate when it comes to estimating the cancer cell fraction of an abnormality (i.e., the proportion of tumor cells carrying the abnormality). Recent publications have shown that higher frequency of a high-risk abnormality, such as Amp1q or Del17p, may be associated with worse outcomes, suggesting that, in the future, an abnormality may only be considered high-risk if it crosses a certain frequency threshold. For this reason, it is becoming exceedingly important to accurately estimate the frequency of a high-risk genomic abnormality in patient samples.

To compare the performance of single-cell RNA sequencing to that of WGS in the detection of CNVs and also the estimation of their frequency within the tumor, WGS was performed on BM tumor cell and/or CTC-derived DNA in 20 patients with available single-cell RNA sequencing data. Using Numbat to analyze the single-cell RNA sequencing, it was possible to detect all CNVs found by WGS (sensitivity of 100%; 95% CI: 80-100). Unlike single-cell RNA sequencing, WGS consistently overestimated subclone frequency. In tumors with large subclones of >85%, it identified them as clonal (FIG. 9). Interestingly, one case in which single-cell RNA sequencing detected Del13q at ~90% frequency, WGS detected it at ~10% frequency, which was a mistake due to the low tumor purity (~16%) of the BM tumor sample.

The experimental results in this example indicate that a multinomial logistic regression model for the analysis of single-cell RNA sequencing data may outperform WGS in accurately estimating subclone size in multiple myeloma. Thus, relative to WGS, single-cell RNA sequencing may have greater utility in prognostication and patient management.

### Example 6. Minimum Number of CTCs

This example illustrates that as few as 10 CTCs are sufficient to correctly classify 90% of patients suffering from plasma cell dyscrasias at least 70% of the time, when using the multinomial logistic regression model of Example 3.

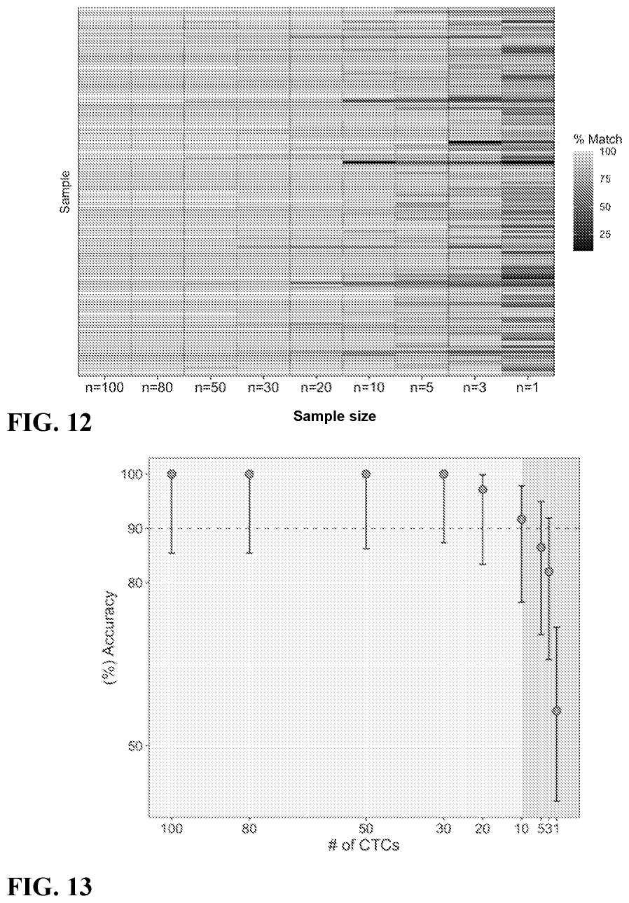

To determine how many CTCs need to be profiled for successful cytogenetic classification, a range of tumor cells (1-100) were randomly downsampled from all CTC samples with 10 iterations per sample size and used as a model to classify samples. Using the multinomial logistic regression model of Example 3, performance deterioration was assessed by calculating the proportion of iterations per sample size for which the sample was classified the same way as when using all available CTCs. As expected, deterioration of performance was observed with decreasing number of CTCs. Surprisingly, the overall performance remained strikingly good with as few as 10 or 5 cells. FIG. 12 shows a heatmap of the proportion of correct classifications out of 10 iterations for each sample size in the CTC cohort, demonstrating increased success at classification using higher sample sizes (as expected), with good accuracy still achieved with sample sizes of 5 or 10.

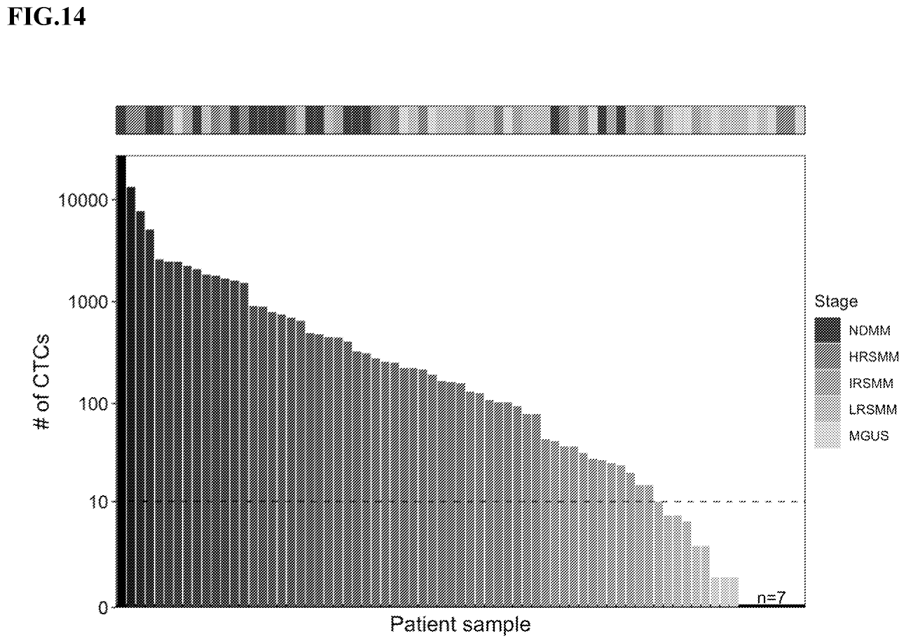

To assess the impact of cell number on the classifier's accuracy, the classification was considered to be a match if it matched the one achieved by FISH at least 70% of the time. Remarkably, 10 CTCs were enough for at least 90% of cases to be classified correctly at least 70% of the time (FIG. 13). This is important because 82% of patients with MGUS, SMM, or NDMM had at least 10 CTCs detected, including 100% of patients with NDMM and 92% of patients with SMM (FIG. 14). Therefore, this approach is capable of correctly classifying most patients with NDMM and SMM for whom such classification may impact patient outcome and management.

This example illustrates that the multinomial logistic regression model of Example 3 is capable of correctly classifying 90% of patients suffering from plasma cell dyscrasias at least 70% of the time, using single-cell RNA sequencing from as few as 10 CTCs.

### Example 7. Improved Multinomial Logistic Regression Model

This example demonstrates that the accuracy of the multinomial logistic regression model of Example 3 can be further improved by expanding the cohort on which the model is trained and updating the genes on which the model relies.

### Expansion of Samples

The patient cohort referred to in Example 1 was expanded to a total of 269 individuals, including patients with MGUS, SMM, and MM, as well as healthy donors, with available bone marrow samples. This allowed the size of the training cohort to be expanded and in turn improved the accuracy of the multinomial logistic regression model for cytogenetic classification as described in Example 3. Specifically, the size of the training cohort was expanded to 134 bone marrow samples from patients classified via FISH or WGS, increasing both the types of classes represented and their respective representation: HRD, n=54; t(11;14), n=41; t(4;14), n=17; t(14;16), n=11; t(14;20), n=7; t(6;14), n=3; t(8;14), n=1.

### Malignant Plasma Cell Identification

Plasma cells were identified and clonotyped as described in Example 1 (see “Plasma cell identification and clonotyping” section). Clones were sorted based on their cell count and candidate expanded clones were identified as clones with: (1) more than 20 cells; and (2) that were at least twice the size of the next clone in order of frequency.

Candidate expanded clones were manually reviewed to determine whether they were normal or malignant. An expanded clone was considered malignant if one or more of the three criteria described in Example 2 (see “Clone identification” section) were met.

Consensus clonotypes were determined for each tumor by sorting malignant clonotypes based on the number of tumor cells using them and selecting the top clonotype. This was done because a single tumor can present with variants of a malignant clonotype (for example, a light chain only variant of the main clonotype). For each consensus clonotype, the Levenshtein distance between each clonotype detected in the sample and the consensus clonotype was computed on the CDR3 AA sequence and normalized by the length of the longest of the two sequences. Clonotypes with at least 90% similarity to the consensus clonotype were considered variants of the malignant clonotype and were annotated as malignant, whereas clonotypes with at least 60% but less than 90% similarity were considered suspicious and excluded from the normal plasma cell compartment.

This approach was used for 231 patients who had single-cell BCR V(D)J sequencing data available. For samples without such data (n=38), malignant cells were identified on the basis of their: (1) clustering separately from normal plasma cells in the RNA space, (2) demonstrating near-monoclonal expression of the patient's tumor's immunoglobulin isotype (detected via serum IFX), (3) consistent expression pattern of key expression markers of plasma cell malignancy, such as key MM oncogenes (CCND1, MAF, WHISC1, etc.) and key immunophenotypic markers (NCAM1, CD19, PTPRC, etc.), and/or (4) presenting somatic copy number variants, as inferred using Numbat(see below).

A total of 205 patients in this cohort had tumor cells detected, and four patients had two distinct tumors detected each, with different malignant clonotype for each tumor, for a total of 209 tumors.

### Copy Number Analysis

CNVs were inferred using Numbat (v1.1.0), as described in Example 2 (see “Determining copy number variants” section), except that CNVs were inferred on tumor cells only for each tumor to increase detection sensitivity. Additionally, data from patients with >15,000 tumor cells (n=14) were downsampled to 15,000 prior to running Numbat for efficiency, and Numbat was run with max_iter=2, tau=0.1, min_cell=10, to further increase detection sensitivity.

Numbat clones with <100 cells were filtered out. Clone-level CNV probabilities were then computed as the median joint probability for each CNV across cells in the respective clone and CNVs were called if the median probability for the event in that clone was >0.65. If the event was deemed to be present (>0.65) in the clone immediately prior to the given clone, the event was called if the median probability in the given clone was >0.55. Variants not called in any clone were removed from consideration and clones with identical CNV profiles were collapsed together.

Numbat CNV calls were then translated into focal, arm-level, or chromosome-level calls. To do this, for each segment, the overlap with both the p and q arms of the corresponding chromosome was computed. If the overlap was >0.85, the event was called an arm-level event for that particular arm. Otherwise, the event was called a focal event. If both arms of a particular chromosome were involved by a single event at the arm level, the event was called a chromosome-level event. If a tumor had at least two trisomies or one tetrasomy, the tumor was considered to be hyperdiploid (HRD). Three tumors with multiple arm-level gains, but which did not meet the above criteria, and had no IgH translocation, were force-called hyperdiploid.

### Cytogenetic Classification

Cytogenetic classification was performed essentially as described in Example 3, with some minor modifications. Briefly, all cells were scored for key MM gene signatures (CD-1, CD-2, MS, MF, HY) reported by Zhan et al. (supra-see Table A) using Scanpy's (v1.8.2) score_genes function. After min-max normalizing each signature across cells, median signature scores were calculated within tumor cells from each compartment in each patient. Median signature scores, as well as mean log1p-transformed gene expression values for key MM oncogenes (CCND1, CCND2, CCND3, NSD2, FGFR3, MAF, MAFA, MAFB, ITGB7) within tumor cells per patient and compartment, were used as features in a multivariate logistic regression model to detect IgH translocations. CCND3 and MAFA were included because they correspond to t(6;14) and t(8; 14) translocations, respectively. ITGB7 was included because it is consistently upregulated in t(14;16), t(14;20), and t(8;14) translocations. These features used to detect IgH translocations in the model were selected based on established knowledge about their association with underlying IgH translocations in MM.

Mean gene expression at the tumor level was used as a feature to overcome drop-out phenomena, whereby a given gene, particularly a gene expressed at low levels such as a gene encoding a transcription factor that is upregulated as the results of an IgH translocation, may not be detected in all of the cells that actually express it. Translocation partner genes were complemented by median signature levels of key cytogenetic signatures (see Table A) to further reduce the reliance on single genes. For translocations of MAF transcription factors, such as t(14;16), t(14;20), and t(8;14), ITGB7 mean expression levels were included in the model. This is because there are cases where little to no expression is detected for the underlying MAF transcription factor, yet ITGB7 is still highly expressed. For tumors with t(4;14) translocation, both NSD2 and FGFR3 levels were included in the model, as well as median levels of the MS signature (see Table A), because FGFR3 expression is lost in a fraction of t(4;14) tumors.

Bone marrow tumor cells from tumors classified by FISH (n=105) or research-level WGS (n=85) were used to train the model iteratively following a leave-one-out approach (n=134). Hyperdiploid cases were used merely as controls for the model to detect the presence of one of six key IgH translocations: t(11;14), t(4;14), t(14;16), t(14;20), t(6;14), or t(8;14).

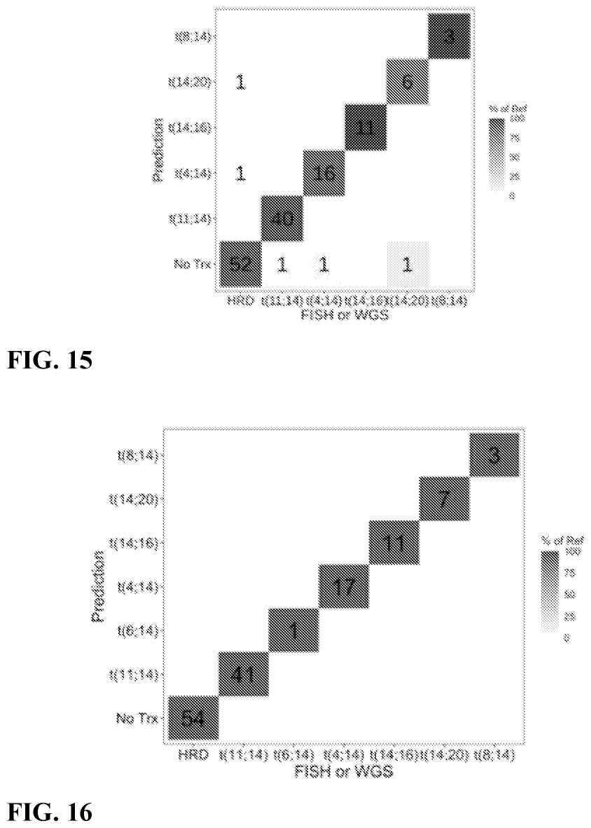

To limit overfitting, leave-one-out cross-validation was performed on the aggregated results from the 134 held-out samples. Remarkable performance was observed, with 129 out of 134 cases being classified correctly (96.3%; 95% CI: 91-99). These results are shown in FIG. 15, a confusion matrix visualizing the performance of 134 leave-one-out models on the 134 hold-out cases. Within each tile, the absolute number of cases is visualized.

Coefficients from the 134 leave-one-out models were aggregated by taking the median and used to generate an aggregate model, whereby the probability for each class is computed using the equation as described in Example 3. Subsequently, the tumor is classified as the class with the maximum probability, provided that probability is larger than 0.5. Alternatively, it is classified as “No Trx” (no translocation).

Following manual review, two tumors with exceedingly high CCND2 expression but without any features of t(14;16), t(14;20), or t(8;14) translocation were force-called t(12;14). One tumor was force-called t(6;14) due to high CCND3 expression. One tumor with high ITGB7 expression but no MAF, MAFB, or MAFA expression was force-called “No Trx”. Three tumors with discordant results in the bone marrow and peripheral blood were reconciliated by following either the bone marrow-based (n=2) or the blood-based (n=1) classification. Tumors classified as having no IgH translocation (“No Trx”) were classified as hyperdiploid (HRD) when Numbat detected at least two extra chromosomal copies in any of the autosomes. Tumors without an IgH translocation according to the classifier or HRD according to Numbat were classified as “Other” (n=16).

Applying this aggregate model on single-cell RNA sequencing data of matched CTC samples, all cases were categorized correctly, giving an accuracy of 100% (n=134; 95% CI: 91-99; see FIG. 16).

This example demonstrates that the accuracy of the multinomial logistic regression model of Example 3 can be further improved by expanding the cohort on which the model is trained and updating the genes on which the model relies.

### Example 8: Multinomial Logistic Regression Model is Superior to FISH

This example demonstrates that the aggregate multinomial logistic regression model as described in Example 7 is superior to FISH in classifying patients suffering from plasma cell dyscrasias such MGUS, SMM, and MM.

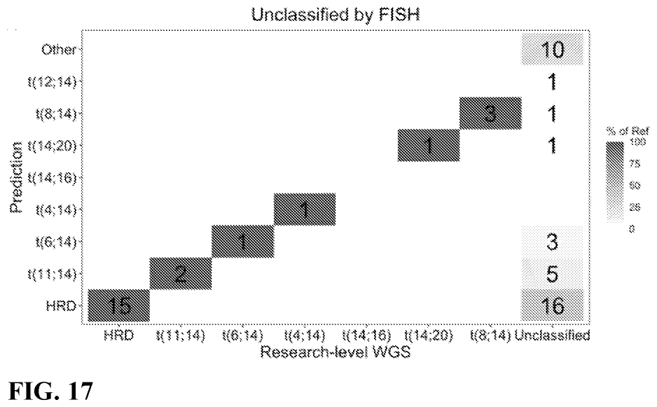

To demonstrate the capacity of aggregate model to improve on FISH, which is the current gold standard in clinical practice, the model's performance was assessed using the 60 primary tumors that were unclassified by FISH. 23 of these tumors had research-level WGS data, while 37 were completely unclassified (FIG. 17)

As shown in FIG. 17, the model correctly classified all 23 cases with WGS data which were part of the training cohort (no patient in these 23 cases had the t(14;16) translocation). Furthermore, an additional 27 out of the 37 remaining cases were successfully classified with either HRD or an IgH translocation: HRD, n=16; t(11;14), n=5; t(6;14), n=3; t(14;20), n=1; t(8;14), n=1; t(12;14), n=1; Other, n=10. Of the remaining 10 cases which were classified as “Other”, 8 cases had CNVs detected, with only 2 tumors predicted to have no Trx or CNVs.

Therefore, the model successfully classified at least 35/37 tumors without either FISH or research-level WGS data (95%, 95% CI: 81-99), demonstrating its superiority to FISH. This significant improvement over FISH is enabled by the superior sensitivity of this approach in detecting tumor cells and its high accuracy in predicting cytogenetic classes.

All publications and other reference materials referenced herein are hereby incorporated by reference in their entirety. Reference to these materials does not constitute an admission that any of these documents forms part of the common general knowledge in the art.

It should be understood that the particular embodiments presented herein are given by way of illustration only, not limitation. Other features, objects, and advantages are apparent from the above detailed description, drawings and examples. Various changes and modifications will be apparent to those skilled in the art.

TABLE A
Gene expression signatures disclosed in Zhan et al.

| MS | PR | LB | HP | CD 1 | CD2 | MF |
|---|---|---|---|---|---|---|
| signature | signature | signature | signature | signature | signature | signature |
| NSD2 | GAGE2A | PHACTR3 | GNG11 | KLHL4 | MS4A1 | CX3CR1 |
| FGFR3 | PCLAF | RASGRP1 | ISL2 | ADRB1 | VPREB3 | TMEM37 |
| BACE2 | CCNB2 | NAMPT | TNFSF10 | INHBE | DTX1 | NUAK1 |
| DSG2 | GAGE5 | PLXNA1 | EPB41L4A- | RBP1 | CLLU1 | GPM6B |
| | | | AS1 | | | |
| CLEC11A | TYMS | PARP15 | SCYL2 | CLEC2D | SORT1 | TLR4 |
| AGAP1 | FANCI | PLEKHA7 | GTF2F2 | SLC7A11 | PAX5 | ITGB7 |
| MYRIP | FAM72A | KCNJ11 | RPL37 | G0S2 | SRC | SMARCA1 |
| FZD2 | NUF2 | COL9A2 | FCHSD2 | MFAP4 | SDC2 | ARID5A |
| SERPINE2 | DEPDC1B | EDN1 | CCRL2 | GPR63 | METRNL | NTRK2 |
| NAP1L3 | GAGE4 | PLEKHA6 | PAK1 | ASB2 | BEND5 | FAM174B |
| FAM171A1 | RRM2 | CSF2RB | SIDT1 | TOX2 | CNR1 | SPP1 |
| JAM3 | UBE2T | CYBRD1 | ELOVL7 | FYN | APLP2 | MCTP1 |
| RUNDC3B | GAGE1 | NTHL1 | F2R | ALPK2 | FCRLB | GREB1 |
| DSEL | KPNA2 | MAP4K3 | ATP5MG | PTGER2 | NF1 | TMPRSS15 |
| BEX3 | MAGEA6 | PPP1R14A | CNTN5 | TBC1D16 | PNOC | DPYSL2 |
| TRIM58 | RFC4 | PCGF5 | DELE1 | CTH | CD27 | COTL1 |
| FZD8 | MAGEA3 | SCUBE1 | FRZB | EIF4EBP1 | PLPP3 | SLC25A20 |
| PBX1 | NUSAP1 | CTTN | EPHB1 | TM6SF1 | MARCKS | CCDC86 |
| PCSK5 | BUB1B | NA | GBA3 | PCDH20 | LAMA2 | NA |
| CLGN | BIRC5 | MAST4 | SNHG7 | CEBPB | PLXNB2 | DEFB4A |
| RAI2 | MCM10 | NA | THG1L | TMC6 | CNPPD1 | SHMT1 |
| TMEM176A | TSR1 | CGREF1 | RBM4B | HADH | CD79A | MFAP2 |
| PPM1H | KIF14 | P2RX5 | DDX58 | ZNF70 | RCBTB2 | TRIM47 |
| ZNF503 | TPX2 | FLNB | NOP53 | PCK2 | PHLPP2 | WHRN |
| CST3 | RACGAP1 | BOC | FL11 | GPT2 | ATP2B1- | CEBPD |
| | | | | | AS1 | |
| WWC2 | E2F8 | BIK | CCDC85A | SYTL1 | RNGTT | NA |
| DTNA | SPC24 | USP53 | RPS17 | PHGDH | DYRK2 | RASSF4 |
| RMST | TOP2A | SMAP2 | VPS51 | NID2 | B3GALNT1 | ABHD12 |
| LRP12 | AURKA | HES5 | XRCC4 | XPOT | ZNF395 | CDH1 |
| VWA5A | SPC25 | SLC39A6 | CDV3 | SESN2 | TRAF5 | ARPC1B |
| KLHL13 | CCNB1 | LYPD6B | SEMA4D | NSUN7 | SPAG4 | TMEM255A |
| MITF | PRR11 | MBD2 | RPL13A | ATF5 | ADSSL1 | AGMAT |
| LOC400741 | ASPM | CACNB3 | TRABD2A | RAB33A | PIK3AP1 | CD109 |
| RNF32 | FOXM1 | KIAA0895 | RSAD2 | RAB31 | PDZRN4 | NMU |
| ZNF521 | CCDC34 | COPZ2 | RC3H2 | CLEC2D | ZNF608 | SYNDIG1 |
| CLIC6 | SLC19A1 | PLS1 | CSNK1G3 | RTL5 | RGS13 | TUBB2B |
| NRIP1 | CDK1 | ARID5B | LAG3 | TRIB3 | TGIF1 | TYMP |
| SPRY2 | TRIP13 | SAMD3 | PIP5K1B | BGLAP | NAGK | DOCK4 |
| TCEAL2 | TTK | NA | RPL7A | BATF3 | DUSP22 | CXCL12 |
| SLC22A17 | MAGEA1 | HRK | OAS2 | ERN1 | TNFSF8 | ABLIM1 |
| MYO10 | MPC2 | ADAM22 | CCR5 | MTG1 | RERE | ARHGAP6 |
| PALLD | EZH2 | IZUMO4 | TALDO1 | STC2 | DHTKD1 | RNASE6 |
| TEAD1 | CENPL | ARNT2 | SCAPER | ITGAL | ITPKB | PEBP1 |
| MARK1 | DLGAP5 | ADTRP | IPO7 | PPP2R5E | | ATP13A2 |
| BEND7 | PRC1 | | POLR1D | CSTA | | APOBEC3A |
| TMEM176B | DSCC1 | | AMIGO2 | TMC8 | | PYGB |
| LARP6 | NEK2 | | LAMP3 | TMX4 | | LCP2 |
| MAGI1 | UBE2C | | | | | SFRP2 |
| | SKA1 | | | | | |

1.-26. (canceled)

27. A method for detecting a B-cell malignancy in a subject, comprising:
(a) obtaining single-cell RNA and receptor V(D)J sequencing data generated from one or more cells of a blood sample of said subject;
(b) processing said single-cell RNA and receptor V(D)J sequencing data to identify one or more clonal cell populations in said blood sample;
(c) detecting said B-cell malignancy in said subject, based at least in part on characterizing said one or more clonal cell populations as malignant or pre-malignant; and
(d) administering or selecting a treatment to said B-cell malignancy of said subject based at least in part on said characterization of said one or more clonal cell populations.

28. The method of claim 27, wherein said obtaining said single-cell receptor V(D)J sequencing data comprises performing single-cell B cell receptor or T cell receptor V(D)J sequencing on one or more nucleic acid molecules of said one or more cells of said blood sample.

29. The method of claim 27, wherein detecting said B-cell malignancy consists essentially of characterizing said one or more clonal cell populations.

30. The method of claim 27, wherein said one or more cells of said blood sample are enriched for a cell type comprising tumoral cells using a cell surface marker prior to obtaining said single-cell RNA and receptor V(D)J sequencing data.

31. The method of claim 30, wherein said cell surface marker used for enrichment comprises CD138, CD38, CD19, CD3, CD56, CD117, CD27, CD45, CD28, CD20, CD5, CD23, CD43, LEF1, or any combination thereof.

32. The method of claim 27, further comprising determining one or more copy number variants (CNVs) for said one or more clonal cell populations characterized as malignant or pre-malignant, wherein said determining one or more CNVs comprises determining an expression-based probability and an allele-based probability that each CNV exists in each cell, and calculating a joint CNV probability for each CNV in each cell based on said expression-based probability and said allele-based probability.

33. The method of claim 32, further comprising clustering cells within each malignant or pre-malignant clonal cell population based on said joint CNV probabilities to identify subclones, identifying a CNV profile for each subclone by calculating a median joint CNV probability for each CNV across cells in said subclone, and calling a CNV as present if said median joint CNV probability exceeds a predetermined threshold.

34. The method of claim 33, further comprising determining a size of each subclone by dividing a number of cells in said subclone by a total number of cells within a malignant or pre-malignant tumor cell population comprising said subclone, and using said subclone size for at least one of prognosis and selection of a treatment or therapeutic regimen.

35. The method of claim 27, further comprising determining a consensus clonotype for each identified malignant or pre-malignant clonal cell population by selecting a clonotype based on a number of cells and identifying as malignant clonotypes having at least about 90% similarity to said consensus clonotype.

36. The method of claim 27, wherein a clonal cell population is identified as expanded when said clonal cell population comprises at least about 20 cells and is at least about 2-fold a size of another clonal cell population detected in said blood sample.

37. The method of claim 27, wherein said characterizing said one or more clonal cell populations as malignant or pre-malignant comprises determining expression of a pre-determined set of oncogenes and tumor surface markers specific to said B-cell malignancy, wherein said oncogenes comprise CCND1, CCND2, CCND3, MAF, MAFA, MAFB, ITGB7, NSD2, FGFR3, LAMP5, FRZB, or any combination thereof.

38. The method of claim 37, wherein said tumor surface markers comprise NCAM1 (CD56), CD19, PTPRC (CD45), CD27, CD117 (KIT), CD28, or any combination thereof.

39. The method of claim 27, wherein processing said single-cell RNA and receptor V(D)J sequencing data to identify said one or more clonal cell populations comprises:
(i) identifying cells expressing one or more lineage markers of a cell type comprising tumoral cells, and
(ii) identifying among said cells expressing said one or more lineage markers one or more expanded clonal cell populations based on said receptor V(D)J sequencing data.

40. The method of claim 39, wherein said one or more lineage markers comprise SDC1, CD38, XBP1, PRDM1, IRF4, TNFRSF17, or any combination thereof.

41. The method of claim 27, wherein said B-cell malignancy comprises Multiple Myeloma (MM), Monoclonal Gammopathy of Undetermined Significance (MGUS), Smoldering Multiple Myeloma (SMM), B-cell lymphoma, B-cell leukemia, Waldenstrom macroglobulinemia, plasma cell leukemia, or any combination thereof.

42. The method of claim 27, wherein said blood sample is a peripheral blood sample comprising circulating tumor cells (CTCs).

43. The method of claim 27, further comprising determining tumor burden by dividing a number of malignant or pre-malignant cells by a total number of cells in the population for which single-cell RNA sequencing data was obtained.

44. The method of claim 27, further comprising determining expression of translocation partner genes for said one or more clonal cell populations characterized as malignant or pre-malignant, wherein said determining comprises scoring each cell for expression of one or more key gene signatures and key oncogenes, and calculating a median signature score and a mean oncogene expression score for each malignant or pre-malignant tumor cell population.

45. The method of claim 27, further comprising analyzing said one or more clonal cell populations characterized as malignant or pre-malignant using a statistical model or machine learning algorithm to classify said B-cell malignancy, wherein said statistical model or machine learning algorithm associates said one or more clonal cell populations with one or more chromosomal translocations comprising t(11;14), t(4;14), t(14;16), t(14;20), t(8;14), t(6;14), or any combination thereof.

46. The method of claim 27, wherein said method is performed on a plurality of blood samples obtained from said subject at different time points, and wherein said characterizing is used to monitor disease progression or assess an effectiveness of a therapeutic intervention in said subject.

47. The method of claim 27, wherein said treatment comprises an autologous stem cell transplant, optionally in combination with a quadruplet combination therapy or a triplet combination therapy.
